(19) United States
(12) Patent Application Publication
(10) Pub. No.: US 2023/0149091 A1
(43) Pub. Date: May 18, 2023

(54) NEUROSURGICAL DECISION SUPPORT SYSTEM

(71) Applicant: ATLAS MEDITECH, INC, Carmel, IN (US)

(72) Inventors: Aaron Afshin Cohen-Gadol, Carmel, IN (US); Jason Dorris, Lone Tree, CO (US)

(73) Assignee: ATLAS MEDITECH, INC, Carmel, IN (US)

(21) Appl. No.: 18/055,482

(22) Filed: Nov. 15, 2022

Related U.S. Application Data
(60) Provisional application No. 63/280,342, filed on Nov. 17, 2021.

(51) Int. Cl. A61B 34/10; G06T 19/00; G06T 15/08; G06T 7/55; G06T 7/149; G06T 7/12

(52) U.S. Cl. CPC A61B 34/10; G06T 19/003; G06T 15/08; G06T 7/55; G06T 7/149; G06T 7/12; G06T 2210/41; G06T 2207/30016; G06T 2207/30096; A61B 2034/105; A61B 2034/107

(57) ABSTRACT

A system and method of providing decision support for planning and performing neurosurgical operations. The method includes preparing a three-dimensional model of the tumor, as well as three-dimensional approach volumes representing regions of the brain occupied by at least a portion of the tumor. Initial paths to the tumor through each approach volume may be determined, and each initial path may be automatically modified to minimize distance through the brain, avoid disturbing adjacent brain tissue, avoid hazardous areas, and use available ventricles, fissures, and voids where possible. The resulting path options may then be presented to a user for consideration, and may be used during the procedure.

### BACKGROUND

The present disclosure relates to a system for planning a neurosurgical operation.

### SUMMARY

Disclosed is a neurosurgical support system including software and hardware components that work together to provide comprehensive decision support for planning and/or performing neurosurgical operations such as the removal or biopsy of lesions or tumors, or any structure within the cranium and associated components. The disclosed approach may also be useful for planning or performing operations on other organs or structures such as the heart, lungs, kidneys, vascular structures, and the like. The system optionally comprises multiple hardware and/or software modules or components which can be used either separately or together throughout a procedure on a human or animal patient. Modules include, but are not limited to, modules for preoperative planning, patient and staff education, surgical staff planning, as well as modules for communication with or control of the orientation and navigation of robotic or other surgical instruments, and visualization equipment during the operation.

In one aspect, the disclosed system includes a decision support software tool that allows the user to position a lesion in a virtual representation of a brain in either 2D or 3D space, or both. In another aspect, the disclosed system is optionally configured to accept data indicating the position of the tumor or other structures that may be identified by this, or another system, such as by using segmentation. The disclosed system is configured to determine a list of suggested neurosurgical approaches and dissection paths or routes with the required incisions and craniotomies for the specific size and location of the lesion to reach the lesion surgically. The system is operable to rank and sort the different path options according to multiple criteria in order to determine the best available options for completing the operation while reducing or eliminating harm to surrounding portions of the brain.

In another aspect, the disclosed system determines recommended positioning of a patient and layout of the Operating Room (OR) facilities during surgery. Based on the recommended path and approach calculated in advance, the software is optionally configured to offer a recommendation for the optimum positioning of the patient's body on the OR table, the orientation of the patient's head within a clamp assembly, or other positioning device, the location of contact surfaces of the clamp assembly, and other aspects of the OR facility and personnel. In another aspect, the system may be configured to accept input from a user which may be used to customize the arrangement of the OR facility.

In another aspect, the disclosed neurosurgical system optionally allows a care giver to upload specific patient images and request an electronic consultation from other care givers at any suitable time before, during, or after the procedure. Multiple care givers at one or more institutions and hospitals may be accessible by multiple different care givers to provide advice or assistance.

In another aspect, the disclosed system includes guidance software configured to control the surgeon and one or more robotic arms of the surgical microscope according to the surgical path during the actual operation to perform the microsurgical maneuvers involved. The system is configured to optionally provide for a user to visualize the surgical route such as by using an output device like a screen or exoscope monitor, or optionally through scope binoculars worn by a user, or a heads-up display, or equivalent device.

In another aspect, the disclosed system may be configured to obtain segmented brain structures from images obtained prior to the operation using Magnetic Resonance Imaging (MRI), Computerized Tomography (CT), or by any other suitable means. The system may be configured to use this information to identify important structures and the boundaries of the affected area. The system may then be configured to automatically modify the internal 2D or 3D model of the patient's brain to match what is shown in the images obtained from other imaging systems. This modified model may then include patient specific anatomical structures, optionally labeled or annotated, thus enabling the disclosed system to use the best available patient specific information for planning the procedure. In addition, identification of these patient specific attributes may be used during the operation to alert the surgeon, such as when surgical maneuvers are placing critical structures at risk, or when the dissection plane is close to the tumor capsule.

Further forms, objects, features, aspects, benefits, advantages, and examples of the present invention will become apparent from a detailed description and drawings provided herewith.

### BRIEF DESCRIPTION OF THE DRAWINGS

FIG. 1 is a diagram illustrating one example of the surgical paths that may be determined according to the disclosed system and method.

FIG. 2 is a flow diagram illustrating one example of the actions the disclosed system may perform according to the disclosed method.

FIG. 3 is a flow diagram illustrating one example of the actions the disclosed system may perform to determine an optimum surgical approach.

FIG. 4 is a flow diagram illustrating one example of the actions the disclosed system may perform to calculate surgical paths.

FIG. 5 is a flow diagram illustrating one example of the actions the disclosed system may perform to determine initial surgical paths.

FIG. 6A is portion of a flow diagram illustrating one example of the actions the disclosed system may perform to determine optimal surgical paths.

FIG. 6B is a portion of the flow diagram of FIG. 6A illustrating one example of the actions the disclosed system may perform to determine optimal surgical paths.

FIG. 7 is a flow diagram illustrating one example of the actions the disclosed system may perform to rank calculated surgical paths.

FIG. 8A is portion of a flow diagram illustrating one example of the actions the disclosed system may perform to determine optimal surgical paths.

FIG. 8B is a portion of the flow diagram of FIG. 8A illustrating one example of the actions the disclosed system may perform to determine optimal surgical paths.

FIG. 9 is a flow diagram illustrating one example of the actions the disclosed system may perform to determine patient specific anatomical models.

FIG. 10 is a component diagram illustrating one example of the components of a system of the present disclosure.

### DETAILED DESCRIPTION

Disclosed is a neurosurgical decision support system and method that allows the user to position a lesion in the brain in 2D and 3D space after which the system responds with a list of suggested neurosurgical approaches (with their associated tailored incisions and craniotomies for the specific size and location of the lesion) to reach the lesion surgically. Patients' anatomy and approaches can be viewed in the context of a highly detailed 3D model of a “generic” or “default” brain, or in the context of a model of a brain specific to a particular patient.

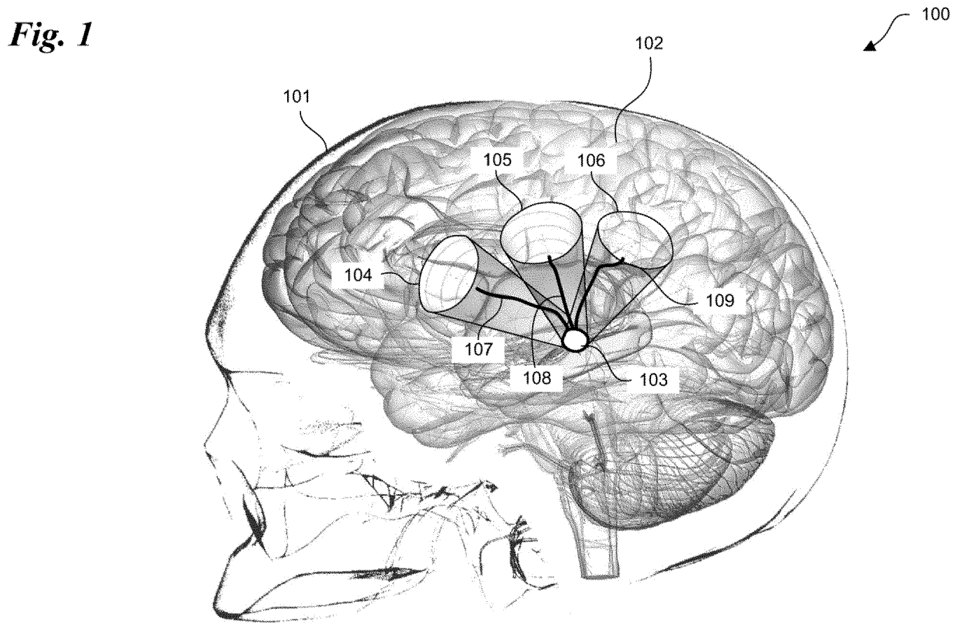

An example of one view of the resulting paths calculated and displayed according to the disclosed method is illustrated at 100 in FIG. 1. At least a portion of the bone structures of a skull 101 are shown with a brain 102 that includes a tumor 103. Surgical approaches 104, 105, and 106 are illustrated here as cones illustrating the top three best approaches determined by the system of the present disclosure according to the disclosed method. The top three surgical paths 107, 108, and 109 optionally pass from separate locations on the surface of the skull 101 and traverse around and through the brain tissue to the tumor 103. The paths 107-109 may be presented to a user in 2D images or as a 3D model allowing a care giver to view the paths from different angles or directions to select the best option for removing the tumor. Surgical instruments may then traverse one of the three paths to remove the tumor thus reducing or eliminating damage to nearby healthy brain tissue. In another aspect, as disclosed in further detail below, the illustration at 100 may be rendered as part of a user interface configured to provide output indicating the best approach for the surgical procedure. This output may be displayed on any suitable display device.

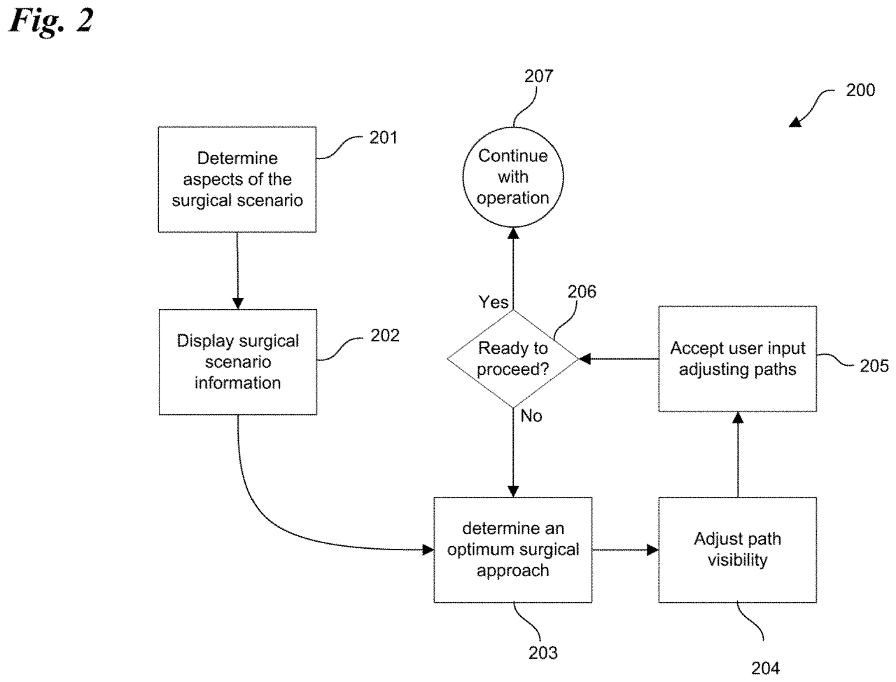

One example of a method of providing decision support of the present disclosure is illustrated at 200 in FIG. 2. In one aspect, the system is configured to determine aspects of the surgical scenario at 201. For example, the system may be configured to accept user input specifying these aspects. In one example, the system may be configured to provide a list of recommended approaches for a given surgical scenario. The system may include one or more display devices that may be configured to provide user interface controls operable to accept input from a user. These controls may optionally be configured to accept input modifying aspects of a surgical scenario. For example, the system may provide options for accepting input selecting a lesion type, shape, size, scale, orientation, or any combination thereof.

In another aspect, the disclosed user interface may be configured to accept input defining the type of tumor present, which may, for example, be chosen from a predetermined list of tumors or any lesion types. This list may include options for accepting input defining certain restrictions such as their shape and positioning as well as their site of origin within the brain or outside the brain. For example, meningiomas are formed along the dura mater (the outermost layer of tissue that covers the brain and spinal cord). In this example, the system optionally accepts input defining an initial categorization of the lesion type which delivers accurate data regarding the surgical approach.

In another aspect, the disclosed interface may be configured to display or otherwise provide detailed information regarding recommendations for surgical scenarios that may be encountered using the system 202. In one example, the information may include recommended paths or approaches specific to a particular surgical scenario. This, or other information, may be included in a database or library of information with data relevant to completing each of a wide variety of surgical procedures. In another aspect, the database or library optionally includes videos, text, and illustrations as well as specific instructions for avoiding known pitfalls or common mistakes so as to provide the best and safest outcome for the patient.

A system of the present disclosure is optionally configured to determine an optimum surgical approach at 203. This optionally includes determining the best available dissection paths or routes for any target location within the brain. These recommended approaches are optionally depicted as linear trajectories that traverse the distance from a starting point such as an area at or near the center of the craniotomy, and extending through the brain to an endpoint such as an area at or near the center of the tumor. These linear trajectories generally traverse a direct shortest path (i.e. “an airplane flight path”) irrespective of brain structures that may be encountered along the way.

The disclosed system may be configured to display the top ranked resulting surgical paths in a user interface window that includes the name of the established approach, along with its rank. Once the optimum surgical paths are determined by the system, a user interface provided by the system may be configured to accept input from a user adjusting the visibility of aspects of the resulting paths at 204 such as the path rankings so that only a specified number of the top scoring paths are visible.

In another aspect, the user interface may optionally provide an editing mode that is configured to accept input from a user to adjust the paths at 205. For example, the user interface may accept user input selecting one or more of the spline knots, or other aspects of a path discussed herein elsewhere, using a pointing device such as a mouse, touch pad, keyboard, touchscreen, and the like. After edits are made, the system may then perform some or all of the disclosed evaluation and ranking actions for determining an operation path that takes into account the user input and determines updated rankings. If the care giver determines that the operation is ready to proceed at 206, the operation may continue at 207.

In another aspect, an optimum surgical approach may include a best case dissection path defining a three-dimensional path through the brain that traverses over, under, and around brain structures that must not, or should not, be disturbed during the procedure in order to safely reach the tumor while traversing the shortest path possible (i.e. “a car ride”).

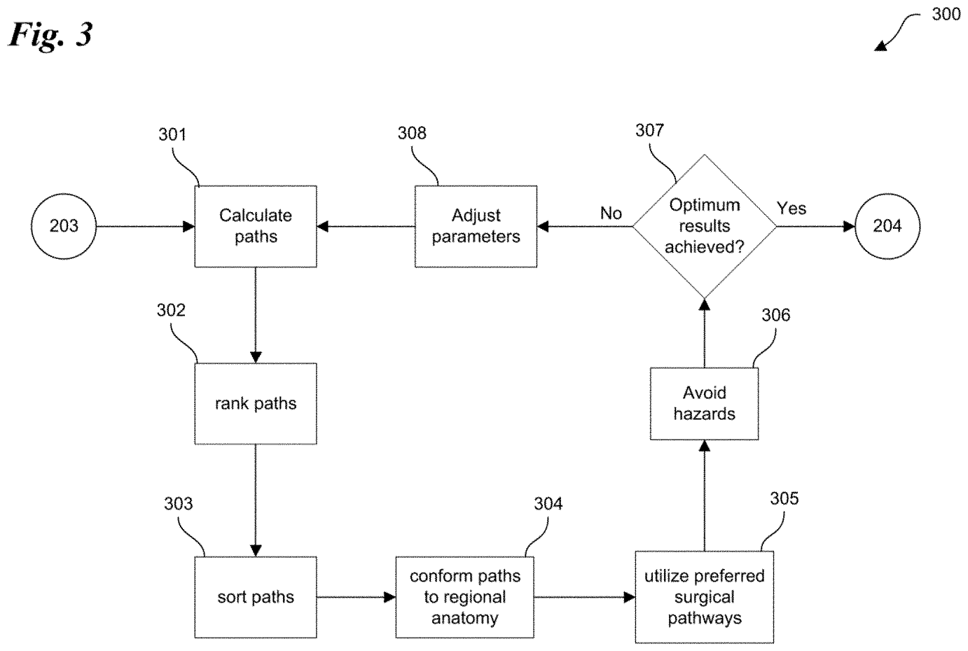

One example of actions the disclosed system may take to determine the optimum paths to the tumor are illustrated at 300 in FIG. 3. Paths are optionally calculated at 301, ranked or scored at 302, and sorted at 303. The user interface may be configured to display the paths to a user optionally sorted at 303 and the sort order may optionally be determined by factors including the shortest path, path curvature, and the like. The approach and/or path may be automatically adjusted to conform to the regional anatomy of the brain at 304. The system is optionally configured to utilize preferred and/or previously described surgical pathways at 305 such as may be defined in the previously mentioned library of information relevant to different procedures. In another aspect, the system may be configured to automatically avoid hazard zones such as highly functional normal brain regions/structures at 306. In another aspect, the system may optionally automatically iterate over the paths multiple times to refine the overall path or approach to achieve the optimum results. If the optimum result is not achieved for a given path, parameters related to the calculations may be adjusted automatically at 308, and the paths recalculated at 301. Examples of these parameters are discussed below in further detail. When the system determines that optimum paths have been determined at 307, the process in FIG. 3 is complete.

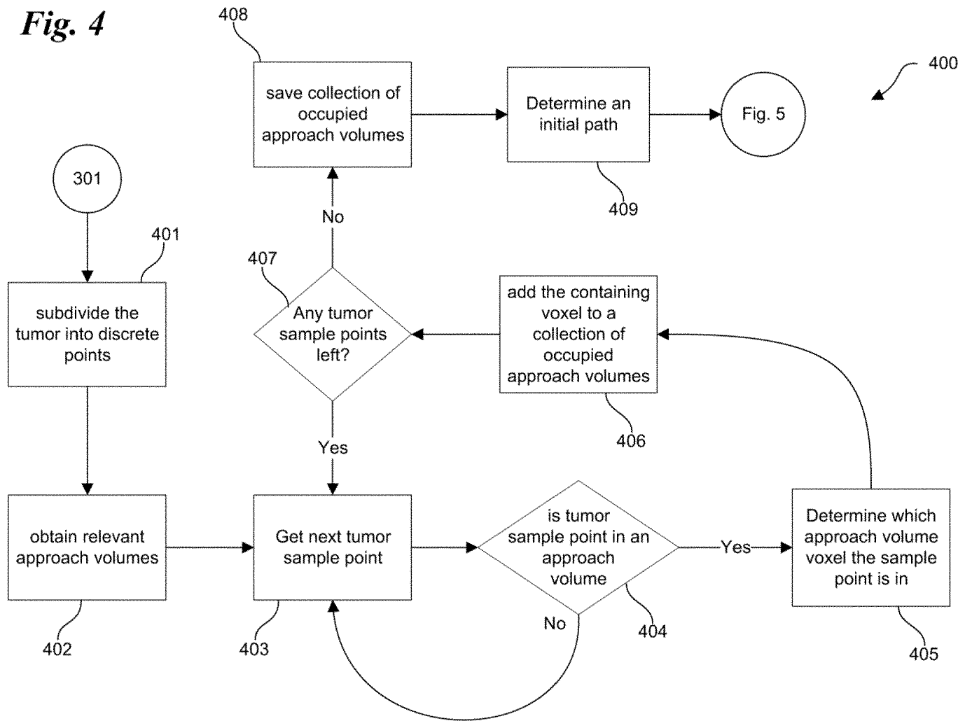

Path calculations at 301 may include numerous individual or iterative calculations with multiple factors considered and weighted in relation to one another. One example of the actions the disclosed system may take in determining an optimum surgical approach and path construction with the necessary path calculations is illustrated at 400 in FIG. 4. In one example, path calculations generally include determining physical attributes of the lesion or tumor such as the location, size, orientation, and shape.

In one example, the disclosed system is optionally configured at 401 to automatically subdivide the tumor into discrete sample points in a predetermined grid. The grid size may be any predetermined interval such as less than 0.1 mm, less than 0.5 mm, less than 1 mm, or optionally 1 mm or more. The spatial location of the sample points is useful for determining if the tumor overlaps a given approach volume. One or more relevant approach volumes may be obtained at 402 and may be used by the system to determine the best overall approach.

Approach volumes of the present disclosure optionally include multiple volumetric labelmaps, each relating to a specific volume of space that is accessible for an established surgical approach. In one example, approach volumes are determined in advance of performing the present procedure by expert analysis of multiple standardized approaches obtained experimentally from hundreds or thousands of previous procedures.

These previously determined approach volumes themselves may be stored within a single channel 8-bit 3D texture format using a nearest-neighbor interpolation, which allows for accelerated texture sampling using a Graphics Processing Unit (GPU) such as might be included in a computing device. At 403, the system may obtain the next sample point for the tumor and determine if the sample point is within any of the approach volumes at 404. If so, the sample point is used to look up the containing voxel within the labelmap for a given approach volume at 405. The system optionally returns a value of 1 if the point lies inside the volume, or 0 if the point is outside the volume. The containing voxel may be added to a collection of occupied approach volumes at 406, and the system may determine at 407 whether there are any remaining tumor sample points left. If so, processing returns to 403 with the next tumor sample point. If none are left to process, the collection of all approach volumes occupied by at least a portion of the tumor is established and optionally stored in a memory at 408. Processing optionally continues with determining initial paths at 409.

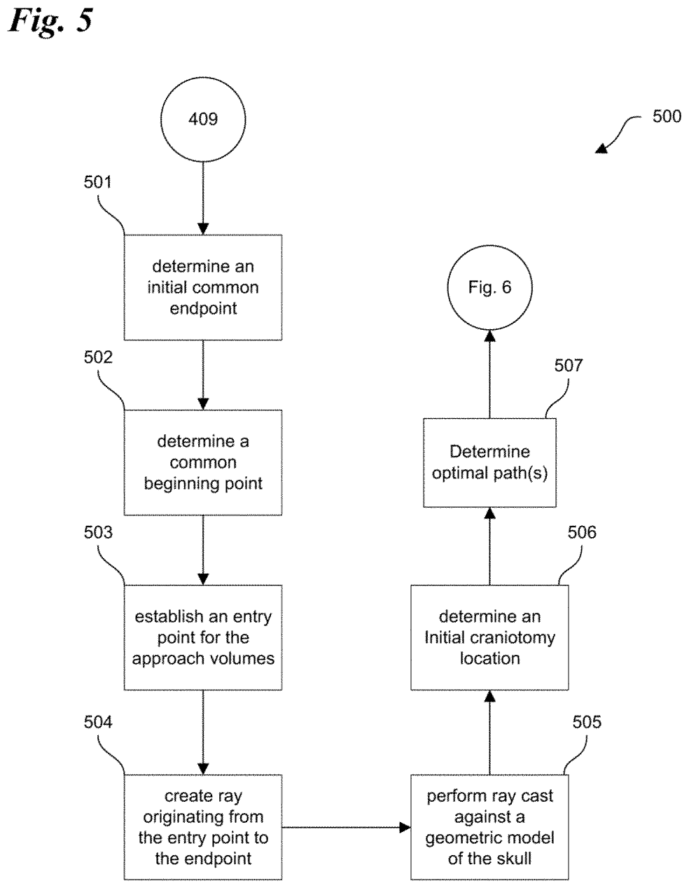

The collection of approach volumes overlapping the tumor may be used as the basis of other approach calculations to consider for a given lesion or tumor scenario. For each of the approach volumes at 408, the system is configured to optionally determine an initial path at 409. One example of the actions the disclosed system may take to determine initial paths is illustrated at 500 in FIG. 5. In one aspect, the system may be configured to automatically determine a common endpoint at 501. In one example, this common endpoint lies at the volumetric center of the tumor. The system may also be configured to automatically determine an initial common beginning point at 502. In one example an initial location is established by referencing a point in space called the Entry Point (EP). The system is optionally configured to automatically pair the approach volumes with one or more entry points at 503. An entry point as used herein generally refers to a 3D point in space around the outside of the skull that provides a general direction of approach for an associated approach volume.

At 504, a ray may be created, originating from the entry point in the direction of the endpoint, such as the tumor center. At 505, a raycast is optionally performed against a geometric model of the skull and an intersection location where the ray intersects the skull may be determined. The location where this ray intersects the skull is optionally used as the initial craniotomy location at 506, and thus the (proximal) beginning point of a particular calculated path. This starting point together with the endpoint determined at 501 defines a line segment that is an initial path representation of the surgical approach.

The initial paths may be modified at 507 to determine one or more optimal paths according to a one or more rules defining predetermined safe dissection paths in the brain. These predetermined safe surgical approaches may involve passing through specific fissures and brain surfaces in creating paths to reach deep lesions while minimizing violation of normal brain tissue. Thus the system of the present disclosure may be configured to modify these initial paths at 507 to arrive at a more favorable optimal path that avoids known problem areas to minimize side effects for the patient.

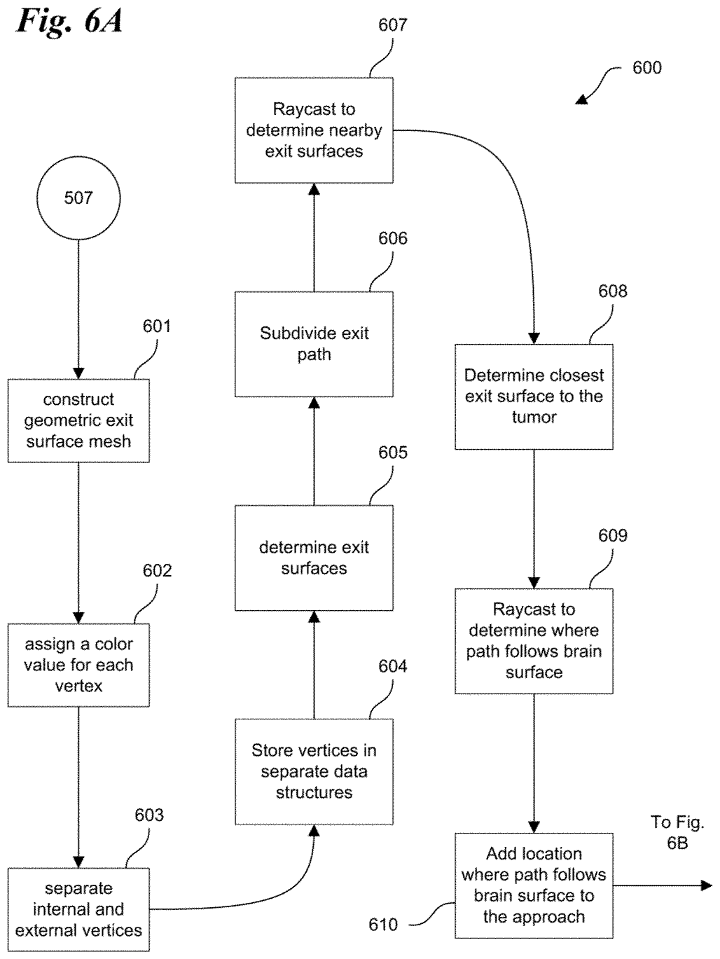

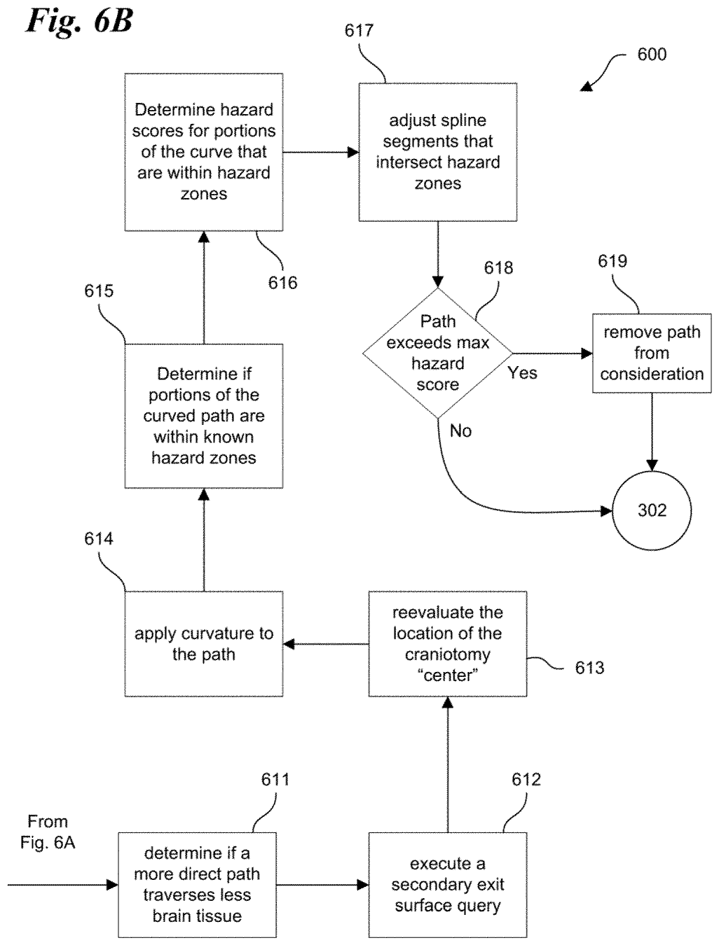

One example of actions the disclosed system may take to automatically determine the optimal available paths is illustrated at 600 in FIG. 6. Geometric exit surfaces are optionally constructed at 601. These exit surfaces define a boundary or surface mesh on which the path can safely travel without entering surrounding brain tissue. The geometric boundary mesh optionally utilizes a 3D geometry feature of assigning and associating a color value for each vertex at 602. For example, a vertex color intensity value of 1 may be used to identify a vertex as an exterior point on the surface, while a value of 0 may be used to identify a vertex as being on an interior point of the surface. Each vertex is optionally collected into separate lists of points for external and internal vertices at 603. These lists of points may be organized in separate data structures at 604. An octree data structure may be utilized for storing the internal and external vertices to facilitate efficient lookup of nearby vertices for a given spatial location. However, any suitable data structure may be used.

Depending on the requirements of the approach, the disclosed system may be configured to automatically determine one or more exit surfaces at 605 when making the optimal path calculations. In one example, such as in the case of an interhemispheric path, only the interhemispheric fissure is considered as a potential exit surface. In another example, paths that are specifically identified as ipsilateral or contralateral may only consider exit surfaces in the corresponding hemisphere of the brain. In other examples, some paths correspond to specific exit surfaces, such as the supracerebellar or suboccipital paths, which use the supracerebellar or suboccipital exit surfaces, respectively. In yet another example, other paths may be free to use any exit surface and test against each exit surface to determine suitability.

Once an exit surface is established at 605, the system is optionally configured to automatically subdivide the path at 606, such as by adding two new points between the start and end of the path. These points may be determined by optionally casting a ray from the tumor center towards the initial craniotomy center. This raycast may be performed iteratively at 607 with an increasing search radius value until exit surface vertices are found to be within the cylindrical volume of the raycast. These points may be sorted by distance to the tumor to find the closest point on the exit surface to the tumor at 608. This point is considered the point along the surgical path where the path leaves the exit surface and enters brain tissue.

In another aspect, the system may be configured to execute an additional raycast at 609 against the octree collection of vertices that are identified as proximally external to the brain. An iteratively increasing search radius may be performed until a collection of points is returned. The system may optionally determine from these points which point is closest to the craniotomy center. This point closest to the craniotomy center may be added to the approach/path at 610. This point represents the location along the surgical path where the path begins to follow the brain surface.

In another aspect, the system may be configured to determine at 611 if a more direct path from the tumor center to the surface of the brain would require less travel through brain tissue. Such a situation may occur, for example, for superficial lesions near the brain surface. In one example, the system may be configured to sample an octree structure containing vertices that lie on the pial surface of the brain in order to find those vertices on the pial surface closest to the tumor. In one example, the system is configured to use the direct path to the tumor instead of the exit surface points when the distance from the tumor to the pial surface is less than the distance from tumor to the exit surface.

In another aspect, the disclosed system is optionally configured to execute a secondary exit surface query at 612 to evaluate the use of intermediate surfaces. This may, for example, include the use of a ventricle that lies between the medial point on the exit surface and the tumor center. The system may be configured to execute a raycast through the octree collection of vertices thereby determining a list of points which may be sorted by distance to the tumor center and the medial point on the exit surface. These two new points are added to the path, and the line segment between them is determined to not traverse brain tissue.

In another aspect, path construction optionally includes reevaluating the location of the craniotomy “center” at 613, which may also be thought as the start of the intradural dissection. At this stage, the exit surface used may not necessarily align with the initial trajectory from tumor center to entry point calculated at 503.

In one example, the system may calculate a ray originating at the tumor center and in the direction of the most proximal point along the path. A point along this ray may be determined at a fixed predetermined distance such as a distance of less than 0.5 meters, less than 1 meter, less than 1.5 meters, or 1.5 meters or more. Any distance sufficiently outside the surface of the skull may be used. The system may be configured to determine another ray from this location back towards the tumor center. This ray may be used to raycast against the skull geometry to determine a more refined location of the start of the intradural dissection or craniotomy “center”, which better aligns with the surgical trajectory of the path. This new craniotomy “center” may replace the original, becoming the first (most distal) point along the path.

In another aspect, the disclosed system defines the surgical path for the corresponding approach as a linear collection of points. Between each point the system defines a line segment that is encoded as being either inside or outside brain tissue. The system may apply curvature to the path at 614 such as by constructing a parametric curve in the form of a B-spline.

In one example, this spline may contain a collection of control points (or “knots”), each consisting of one coordinate for the knot location, and 2 coordinates representing an incoming and outgoing vector of the curve. The line segment connecting these two points is tangential to the curve of the spline at the location of the knot, and is collectively referred to as the “handle” of the knot.

In this example, for each interior point of the path (excluding the first and last point), two new points are optionally determined by linear interpolation of the current knot position and its neighboring knot's position. Spline knots may then be created for each of these interpolated points, with the handle positions optionally determined by linearly interpolating between the knot position and the position of the original point position that the two knots were created from for the inside coordinate, and by linearly interpolating between the knot position and the position of the neighboring knot for the outside coordinate. The parameter used for the linear interpolation between knots may initially be constant throughout the length of the spline, providing handle lengths proportional to the distance between knots. In another aspect, the system may be configured to accept input from a user adjusting the distance between the handle points during path editing. This optionally allows for fine adjustment of curve radius for each knot.

With the spline representation of the path constructed, a new set of discrete points may be created along the length of the spline curve such as at uniform intervals. Each of the points in this set may be used to sample against one or more volumetric label maps such as those obtained from the predetermined library of maps discussed above. The system may thus determine at 615 whether the points lie within one or more of the different volumetric structures in the library.

For example, some of the maps in the library may include labelmaps representing hazard zones indicating areas of the brain that may be highly functional (or “eloquent”), or otherwise especially susceptible to damage by surgical intervention. Some or all of the hazard zones may be assigned a numerical severity score representing the relative danger associated with this volume, relative to other hazard zones. A total hazard score for a given spline may be calculated at 616 by increasing the total hazard score for each point that is found to be within a hazard zone. For example, the spline's total hazard score may be determined by increasing the hazard score by an incremental amount determined by multiplying the spline interval distance by the severity score of the corresponding hazard zone passed through by that interval of the spline.

In another aspect, the disclosed system may be configured to refine the path at 617 by adjusting spline segments that intersect hazard zones. For example, the system may automatically translate the position of individual spline knots with a goal of minimizing the path's total hazard score.

In another aspect, the system may also adjust the path to minimize the path length. This may be done by sampling a point along the spline that intersects the hazard zone labelmap, and then optionally sampling this same location within another volume that contains a composite of all hazard zones. This composite volume represents a signed-distance field, which contains a floating point value that relates distance from the center of the voxel to the nearest surface of a hazard zone, such that values inside a hazard zone have a negative value, and values outside the hazard zone have a positive value. The system is configured to automatically determine a gradient direction leading away from the surface. This may be done, for example, by sampling the neighboring voxels. In another aspect, the system may be configured to determine the resulting cumulative hazard score for the path, as well as total path length and curvature, and provide parameters for a gradient descent algorithm that steers the path adjustment towards favorable conditions. This may be determined by, for example, iteratively moving the spline point along this direction, away from the hazard zone.

An additional filtering process at 618 evaluates the path's hazard score against a maximum hazard threshold. The system optionally invalidates a path and removes it from a list of recommended paths for the user when a path exceeds this threshold. In this way, the severity value associated with each volume may be used as a factor for determining how far the path can move through nearby brain structures before the path is invalidated. After this process is completed for each approach volume and paths that overlap the tumor, a collection of fully constructed valid paths is available for evaluation and ranking.

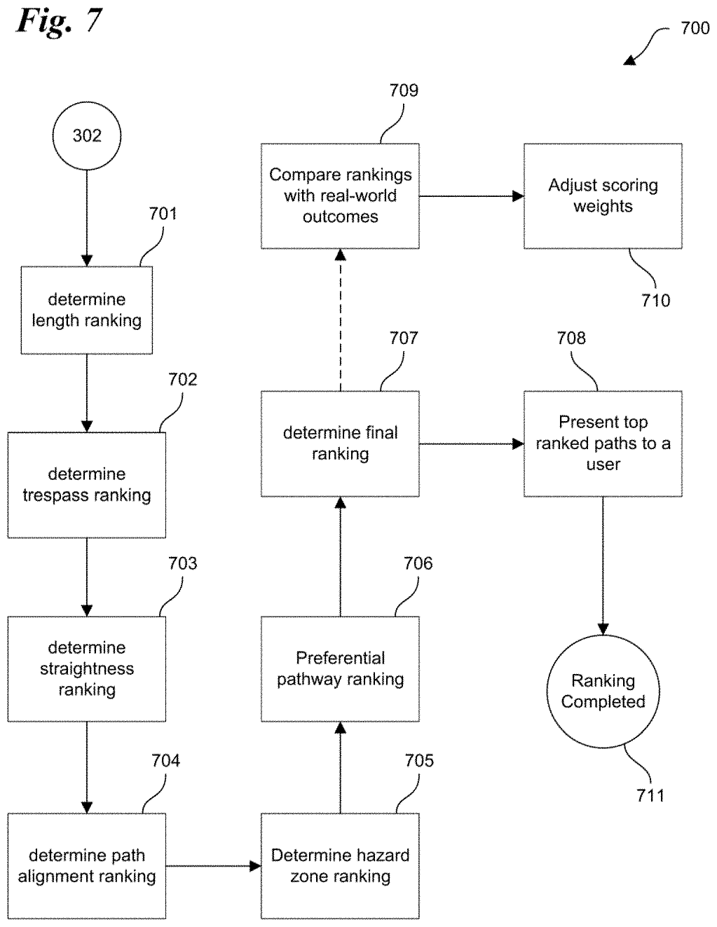

The system may be configured to score or rank the surgical paths at 302 according to one or more scoring criteria. The scores may be stored as normalized floating point values varying between 0 (worst) and 1 (best). The criteria scores or rankings may then be weighted for each criteria by a value that represents the importance of this criteria relative to other criteria. All criteria scores for each path may be summed and the paths with the highest total scores are optionally presented to the user as recommended paths. One example of actions the disclosed system may take to determine rankings for different paths is illustrated at 700 in FIG. 7.

In one aspect, the disclosed system may be operable to calculate a total path length ranking at 701. The total path length optionally measures the distance along the path, from the craniotomy center to the tumor center. For each path calculated, the system may be configured to determine the length of each path segment for a given path and optionally to add these length values together to determine the length of the entire path. In ranking the path by length, the system may be configured to assign a higher value to paths with a shorter total length and a lower value to paths with a longer total length.

In another aspect, the disclosed system may be configured to calculate a path trespass ranking at 702. The path trespass ranking measures the total cumulative distance along the path where the path passes through brain tissue. The trespass ranking optionally considers the full path from the entrance into the pial surface to the tumor center. The path trespass calculation optionally excludes areas of the path that maneuver through fissures and/or grooves or furrows (i.e. sulci) in the brain tissue. It also excludes portions of the path that travel through hollow portions or cavities in the brain (i.e. ventricles). In this instance, the system ranks paths with a shorter length higher and as more favorable while paths with a longer length are given a lower, less favorable, score.

System of the present disclosure is optionally configured to calculate a path straightness ranking at 703. In one example, the angle of alignment between each path segment is optionally calculated and then added together for the entire path. Thus paths with segments that are closer in angular alignment (more straight) obtain a higher score, While paths with more variability in the angular alignment (more crooked) are optionally assigned a lower score.

At 704, the system may be configured to calculate a path alignment ranking. To obtain a path alignment ranking, the system optionally compares the path with the alignment of the longest longitudinal axis of the tumor. This may be formed by, for example, calculating the angle between the path direction and the tumor direction. In this instance, paths that more closely align with the longitudinal axis of the tumor are given a higher score because paths or approaches along this axis are minimally invasive to the surrounding tissue in comparison to perpendicular approaches/paths. Thus, paths that are more perpendicular to the longitudinal axis of the tumor may be assigned a lower score.

At 705, a system of the present disclosure may optionally calculate a hazard zone ranking based on the level of severity of hazards traversed by the present path. As discussed herein elsewhere, different volumes of the brain may be characterized according to the risk posed to the patient if those individual areas are disturbed. Each of these regions may be assigned an associated floating-point value that represents the severity of the risk. The length of any path segment that occupies one of these regions may be multiplied by the risk severity value. These values may be added together by the system to obtain an overall ranking of hazards likely to be encountered if the procedure follows the given path. Thus lower values indicate reduced hazard to the patient, while higher values indicate increased hazard.

In another aspect, the system of the present disclosure may determine a preferential pathway ranking at 706. This ranking may be thought of as the inverse to the concept of “hazard zones”, where 3D volumes are constructed that represent regions of the brain that are more favorable candidates for surgical paths. In one example, regions of the brain may be associated with a floating-point value that scales with preference. The system may be configured to multiply the length of a line segment by the preference value to determine an overall preference ranking for each line segment. These values may then be added together to achieve an overall preferred pathway ranking. Higher values may thus indicate a more preferred pathway, while lower values may indicate a less preferred pathway.

After scoring of each path is complete, the system is optionally configured to determine a final ranking at 707. This final ranking may, for example, be determined by summing together the scores of the individual rankings 701-706 for each path under consideration. The system may be configured to rank the paths by sorting the scores from highest to lowest. In one example, the system may present the top ranked path, the top three ranked paths, or more, to a user as recommendations. These top-rated paths may be rendered graphically to a user interface using a display device. One example of the resulting imagery is illustrated in FIG. 1 and discussed above.

In another aspect, the system may be configured to present top ranked paths to a user at 708. This presentation may appear similar to what is shown in FIG. 1. In another aspect, presentation may include a 3D geometric representation of the spline curves for each path such as in the form of an extruded curved line with a circular cross-section. The system may also optionally provide texture mapping UV coordinates for each vertex of the path and may also optionally apply a texture map of the animated Y offset thus creating the visual impression of motion. In another aspect, path segments may be color-coded to identify which segments are outside the brain, along an exit surface, or within brain tissue. A corresponding label may be used to identify each path by its rank. Additional markers may be placed along each path at each interval that overlaps a hazard zone, with a color-coding relating to the severity classification of the hazard zone.

In another aspect, the disclosed system may be configured to use Machine Learning (ML) to optimize and validate path scoring. In one example, the above mentioned scoring criteria generally includes a corresponding weighting value associated with its importance relative to the other criteria. These score weighting values may be adjusted to prefer paths that score higher with the related criteria. To optimize and validate these weighting values, the software optionally employs machine-learning techniques to compare different weighting scenarios against real-world datasets at 709. These real-world datasets may be collected from any suitable source, such as, for example, from records of experiments and procedures performed on actual human or animal patients. In one aspect, a collection of case-studies may compare patient-specific tumor locations against the actual surgical approach employed by the surgeon.

For each case-study, the disclosed system may be configured to use an identical tumor position and to compare the highest-rated paths to the actual path used by the surgeon. A machine-learning algorithm may then be employed to then adjust the scoring weight values for some or all of the criteria at 710 in order to generate top-rated paths that are closer to the case-study paths. This determination may be repeated for each case-study and statistical analysis employed to determine optimal criteria weighting values that produce results that best match real-world decisions made by skilled neurosurgeons. In another aspect, the disclosed system may employ this approach in order to validate surgical paths recommended by the software against actual patient outcomes for the same or similar procedures.

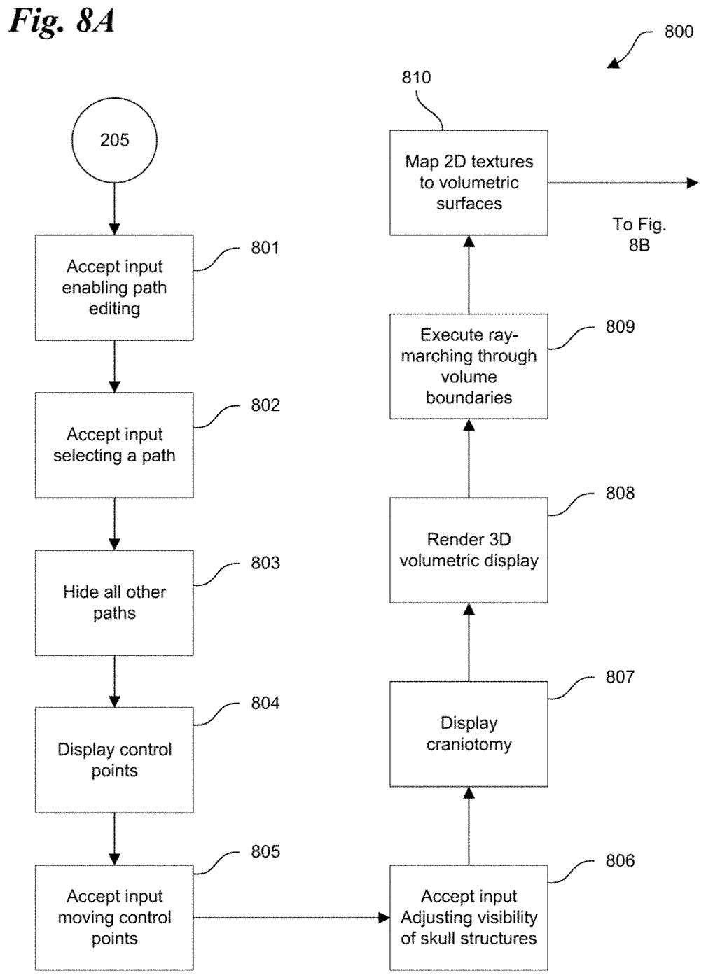

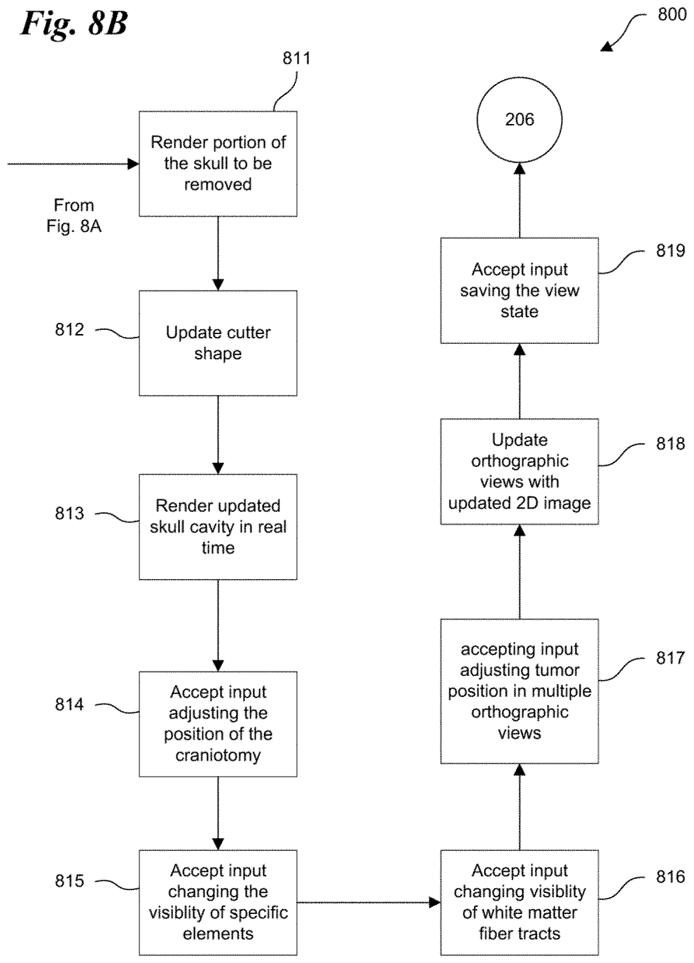

In another aspect, the system of the present disclosure may be configured to provide manual path editing options as mentioned in FIG. 2 at 205. This option may, for example, be offered to a user after path construction, refinement, and ranking are completed according to the present disclosure. One example of actions the disclosed system may be configured to take to allow for path editing are illustrated in FIG. 8 at 800.

The system may be configured to accept input from a user adjusting a surgical approach or path, such as after the approach has been automatically determined according to the disclosed method. For example, the user interface may set input enabling a path editing mode at 801. When enabled, user input selecting a path to edit is accepted via an input device at 802, and all other recommended paths are optionally hidden from view at 803. The system may then display control points along the selected path to a display device, preferably in a way that allows the system to accept user input interacting with the control points. In one example, the system may be configured to accept input from a user selecting and moving (e.g. “clicking and dragging”) a control point. Put another way, the system may accept input from a user moving a control point along an XY plane aligned parallel to the present field-of-view and perpendicular. In another aspect, the user interface may be configured to “snap” these control points to an associated exit surface, thus preventing the adjusted path from entering brain tissue.

In another aspect, the system may be configured to accept input adjusting the distance between handle points for knots associated with one or more control points. By doing so, the handle size may be scaled outwards from the control point which allows the system to accept input defining fine adjustments to the curve radius for each knot along the path without affecting the lateral rotation of the curve around this knot.

The system may also be configured to accept input adjusting the visibility of the skull structures at 806. For example, in path edit mode, a volumetric representation of the skull may be rendered to the screen. The system may also be configured to provide visual indication for the craniotomy opening at 807 for a given path relative to the representation of the skull. In addition to the craniotomy, a visual indication of the recommended incision shape is optionally projected onto the skull for the given approach. The system may employ a 3D shader that accepts 3D texture input containing a high resolution binary label map that has had a 3D gaussian blur applied to generate a volumetric representation of the skull at 808.

In another aspect, for each path fragment, the system may employ a shader algorithm or module operable to perform hundreds of ray-marching steps along a camera ray defining a specific field of view at 809, through the volume bounds. In one example, the system is configured to sample neighboring voxels to establish a gradient that represents the normal direction of the implicit surface. This normal direction is optionally used by the system to calculate a surface color value, such as by using the dot product of the normal and camera direction to simulate diffuse and specular lighting of the surface. The normal direction may also be used by the system to optionally perform tri-planar mapping of tileable 2D textures at 810 in order to provide additional high-frequency detail to the volumetric surface.

The volumetric skull shader module which may be used by the disclosed system optionally accepts a second 3D texture that contains the volumetric shape of the portion of skull to be surgically removed at 811. For example, this texture may be encoded as a signed-distance field with voxel values relating the distance to the surface of the cutter shape. This texture may then relate to a volume with exactly the same bounding box as the skull volume, but can use a different spatial resolution. The shader module employed by the system optionally samples this texture and may then draw any portion of the skull that lies within the cut volume as transparent. Additionally, sampling neighboring cutter voxels provides for a gradient and normal direction in order to provide a realistic rendering of the cut cross-section surface.

In another aspect, the system is optionally configured to accept user input updating the cutter shape at 812 and may be configured to render the resulting cavity in the volumetric skull in real time at 813. This may be done by the system, for example, by constructing a geometric cutter shape with strategically designed topography that allows for several shape variations to be modeled to conform to standard craniotomy shapes. These shape variations may be encoded into the mesh as blend shapes, which parameterize the linear interpolation between the vertex positions in both states. The system is optionally configured to accept input controlling various detailed aspects of how the cutter shape will look, including width, length, depth, roundness of corners, narrow base, etc. This may be achieved, for example, by mixing several of these blend shape parameter weights together.

The system may also be configured to accept input adjusting the position of the craniotomy at 814, such as by allowing the user to select and move the craniotomy in screen-space using a user interface handle. For example, the system may optionally execute a raycast from the handle position to the tumor center, which intersects with the skull surface where the front edge of the cutter shape is placed thus providing a uniform cutting depth from the outside surface of the skull. Whenever input is received specifying a new location for the craniotomy, or whenever input is received adjusting the parameterized cutter shape, the geometric cutter shape may be re-written by the system into the signed-distance field texture and applied to the volumetric skull.

In another aspect, the system may render highly detailed 3D anatomical models used for visualization that are split into numerous anatomical regions. For example, the system may display a user interface that includes user interface controls and keyboard shortcuts for accepting input from a user to change the visibility of individual anatomical elements at 815. These anatomical elements include, but are not limited to, regions of the patient brain, skull, and skin. Each anatomical model may be shaded by the system using a rendering workflow to allow for photorealistic depiction which may be based on physical attributes of the patient.

The system may provide a second “X-ray” rendering mode that if enabled, optionally provides a transparent rendering of the anatomy. This rendering may include a Fresnel intensity falloff with an additive blending mode. In another aspect, the system may be configured to accept input configured to change the visibility of detailed white matter fiber tracts at 816. These white matter fiber tracts may be visualized with or without animated pulsing textures flowing along the length of each tract. The system may also provide additional path drawing controls giving the user options over the visibility of paths, exit surfaces, hazard zones, or other reference features.

In another aspect, the system may be operable to position the tumor in 3D space by accepting input from a user adjusting the position of the tumor within multiple orthographic views at 817. The system may, for example, render a four-panel display showing axial, coronal, and sagittal views, as well as an optional fourth view from a 3D projection. The system may be operable to accept input adjusting the tumor position along a corresponding plane by allowing a user to select and move (e.g. “click and drag”) the graphical representation of the tumor in X, Y, and Z space within each view. The user interface may be configured to provide slider controls in each view operable to accept user input for positioning the slice (and tumor) along the corresponding axis. In another aspect, the user interface may accept input from a user changing the position of the tumor along a plane aligned parallel to the present field-of-view (perpendicular to the camera ray), and at a depth matching the distance of the tumor to the camera. This may be performed, for example, by accepting input selecting and moving the tumor position within the 3D view. As the tumor position is updated, each orthographic view is optionally updated automatically at 818 as the system renders a new 2D view of the anatomy at the corresponding slice. This allows for precise tumor placement relative to anatomical structures.

In another aspect, the disclosed visibility settings are optionally serializable within a data container to allow for saving and loading of the view state at 819. In this way a user's changes may be captured and saved for later reference or for accepting further input making additional adjustments at a later time.

In another aspect, the disclosed method for determining a path and or approach for surgical procedure may be applicable in other procedures such as in the placement of screws in the spine, placement of electrodes in the brain using robotic navigation devices, a stereotactic biopsy procedure, or a stereotactic radiosurgical procedure, to name a few nonlimiting examples.

In another aspect of the disclosed system, the system may be configured to perform the disclosed calculations based on patient specific anatomical data and/or imagery rather than on a generic depiction of human anatomy. In either case, the disclosed system is configured to make comparisons of the tumor location relative to anatomical structures in a 3D brain. This brain may be a generic phantom 3D anatomical model that is appropriate for patients with typical normal anatomy. However, in some cases, individual patient anatomy may significantly differ from the phantom 3D anatomy model used by default by the disclosed system and method. For instance, large tumors may displace portions of the brain, changing the location of key anatomical structures, midline, vessels, or hazard zones, therefore possibly creating operative corridors to the tumor that may not be present in other situations. To provide more accurate evaluation of surgical paths, the phantom 3D model may be adjusted to better fit patient-specific anatomy.

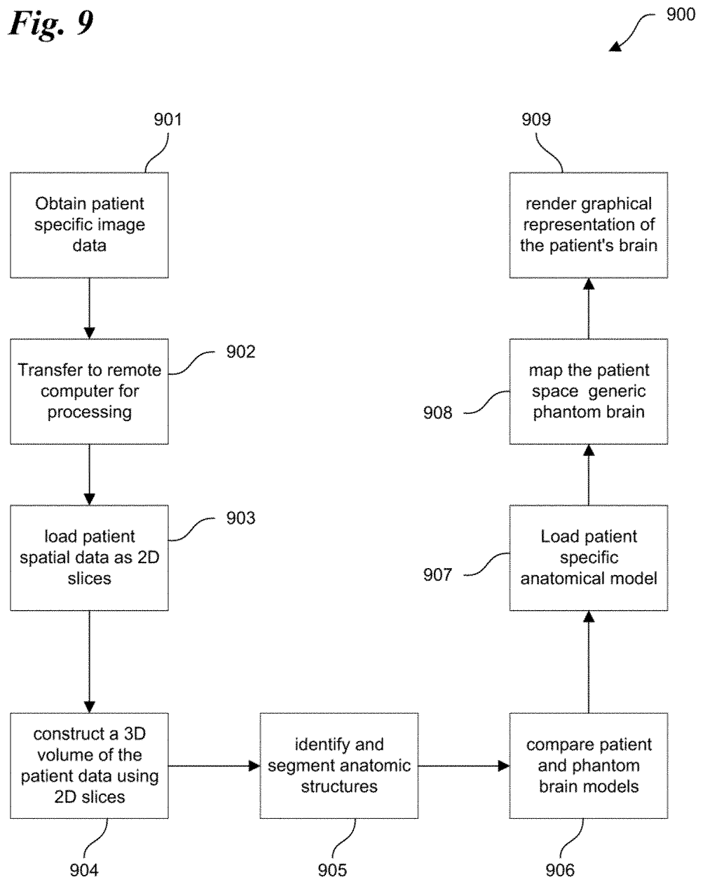

One example of actions the system may take in determining user specific anatomy is illustrated at 900 in FIG. 9. The system of the present disclosure may be configured to provide a user interface configured to present options for obtaining a patient specific anatomical spatial data

In one aspect, patient specific imagery may be accessed by the system via image files on a local computer, network attached storage, cloud storage, via a Picture Archiving and Communication System (PACS), or by other suitable means at 901. The patient images may be acquired in multiple modalities and sequences using MRI, CT, or other suitable imaging technologies. For example, the imagery may be available in any suitable format including MRI, MRI_t1, MRI_t2, MRI_FLAIR, MRI_tic, MRA, MRV, and CTA, to name a few nonlimiting examples.

In another aspect, using patient specific spatial data may involve the use of an advanced processing pipeline to prepare patient data for use in the application. This may involve several sub-processes that require a specific software environment and possibly specialized hardware that may not be available to some users of the disclosed system. To provide processing tasks in a timely manner, in a controlled environment, a remote server optionally handles the workload of conforming patient images for use in the application.

The patient images are optionally de-identified on the local machine, stripping sensitive personal patient metadata from the source images. These images may be optionally transferred from the client computer to a remote server via a communications link to a computer network at 902. This remote server may be in the same room with the client computer, in another room of the same building, in different building across town, or anywhere for that matter. The remote server optionally provides a unique token specific to this processing task to the client software, which may be used later to determine the status of the processing job. The user may also receive an email notification at the completion of the processing task, allowing for work to be continued in a later session.

In one example, the remote processing server executes software configured to load patient-specific imagery at 903 as a series of 2D slices taken at distance intervals along a corresponding axis. The software may then construct a 3D volume of the imagery by relating the X and Y dimensions of the pixels in each image slice to the Z position of the slice at 904. The system may be configured to evaluate these volumetric data sets to identify and segment anatomic structures into labeled 3D volumes at 905. These 3D volumes are optionally compared to corresponding anatomic structures in the phantom 3D brain model at 906 in order to evaluate the difference in displacement between the phantom model and the patient anatomy.

When image processing is complete, the resulting volumetric data may be accessed, such as by downloading it via the computer network. Patient-specific data may be transferred from the processing server to the local client where it may be temporarily stored for the remainder of the session. The system of the present disclosure may then load the patient data at 907, thus providing patient specific visualization for both the full collection of volumetric images as well as 3D mesh representations of anatomical segmentations.

In another aspect, the system may provide an additional volume containing a non-linear grid transform, and may be configured to map the patient space to the space of the generic phantom brain at 908. In one example, each voxel of the grid transform corresponds to 3 displacement values for the X, Y, and Z axis. The system may be optionally configured to combine the X, Y, and Z values to form a displacement vector for this voxel. It may also be configured to sample a point between the voxel centers which optionally results in an trilinear interpolated value of displacement for the position.

In another aspect, the system of the present disclosure may be configured to render a graphical user interface that includes a detailed phantom model that has been modified to conform to the patient's anatomy at 909. In one example, the updated patient specific model is rendered by sampling the world-space position for each vertex in the phantom model in the grid transform in order to determine a displacement offset vector for that position. The mesh is optionally reconstructed with a new set of vertex positions with the displacement offset applied. The result is a deformation of the generic phantom 3D anatomy model that conforms to the patient-specific radiology, allowing for path construction that considers patient-specific anatomy. This newly deformed phantom 3D model of the patient's brain may be referred to herein as the “real brain.”

When using the patient specific spatial model, the disclosed display models are optionally displaced to conform to patient space, but the underlying volumetric reference data (i.e., approach volumes, hazard zones) optionally remains in the original phantom space. The programming functions used to sample these volumes may apply an inverse transform to the sample position, converting it from patient space to phantom space, and return the corresponding value. All other path scoring functions mentioned above may be tailored to the “real brain” or other patient's anatomy to provide a patient specific surgical path to the lesion.

In another aspect, the disclosed system is optionally configured to determine and present to the user a recommended positioning of the patient within the overall context of the Operating Room (OR) setup during surgery. In another aspect, the software optionally provides a recommendation for the positioning of the patient's body on the OR table, the orientation of the patients' head within clamp assembly, as well as a configuration of contact surfaces (e.g. “pins”) of the clamp assembly. These determinations may be made based on the top recommended path or approach determined according to the present disclosure.

In one example, the recommended positioning is one that allows for best surgeon access to the surgical corridor within the OR. A finite collection of patient positions predetermined by expert analysis and experimentation may be made available to the system for the purpose of determining an optimum positioning scenario for specific cranial approaches. When a top-ranked path is determined according to the present disclosure, the patient positioning may be updated to reflect the recommended positioning for the specific cranial approach for that particular path. The patient positioning may be demonstrated in a 3D model of the operating room generated by the disclosed system. Thus a user may be allowed to navigate the virtual operating room and view the recommended positioning for the patient, equipment, and care givers from multiple points of view. In another aspect, the system may be configured to accept input adjusting the position of the OR components according to preferences of a specific caregiver.

In another aspect, the disclosed system may be operable to carry over the tumor location and information include the surgical approaches and paths obtained by the disclosed method to the brain of the patient appearing in the virtual OR setup. The system may be configured to accept input defining the preferred view of the OR include showing or hiding components of the patient's head for a more in-depth analysis of the tumor location in relationship to items and caregivers positioned on or around the OR table.

In another aspect, during the patient's head or body repositioning, the surgeon's position as well as other devices used by the surgeon will move to maintain an ergonomic OR setup to complete the operation. Devices used by the surgeon may, for example, be configured to allow for dynamic movements using an iterative inverse kinematic solver. In addition, the view of the surgeon through the microscope or any visualization tool may be superimposed on the corners of the scene. By refining the location of the surgeon and patient head orientation relative to the microscope alignment, the user may create a surgical configuration that allows for optimal access to the surgical corridor with minimal obstructions.

The system may be further configured to carry the results of the incision and craniotomy calculations determined by the method disclosed herein onto the head of the patient in the OR when the procedure is performed. In addition, the system is configured to determine instruments and products that may be needed for the procedure, and further to display these recommendations in a user interface accessible by a caregiver before and during the procedure. These recommendations include, but are not limited to, surgical tools or products, dissecting instruments, and any other equipment or products necessary to perform the operation effectively. In another aspect, the system may be configured to send the recommended list of tools and products that may be needed to complete the operation to caregivers such as the OR staff of the hospital to allow for seamless preparation before the day of the operation. This information may be sent via any suitable communications link and/or computer network such as by email, Simple Messaging Service (SMS), or other communications systems.

In another aspect, the system of the present disclosure may be configured to provide communication links with other care givers. These communication links may be useful for sharing patient information between care givers, and for obtaining recommendations or other useful input before, during, or after the operation. In one aspect, the system is configured to accept 2D images or 3D models specific to a given patient. The system may store and transmit this information to other caregivers to facilitate discussions regarding the best course of action for treatment. In one example, patient clinical information may be uploaded and the designated consulting care giver may optionally receive an SMS text message, email, or other notification requesting the remote consultation. Multiple hospitals, surgical centers, or other facilities may be linked by secure communication links created and maintained by the disclosed system for the purpose of facilitating care giver collaborations.

In another aspect, the disclosed system may provide a multi-user Virtual Reality (VR) interface by which consultations may be performed within the context of a virtual OR. This VR functionality may be implemented as a stand-alone application configured to render the disclosed 3D operating room environment discussed above, but with the additional aspect of support for use with 3rd party commercial head-mounted displays and tracked controllers. The system may be configured to provide a custom user-interface and interaction system for VR controls and to provide access to the operating room features for adjusting patient position, microscope position, and/or other surgical tools or equipment. In another aspect, the system may be configured to accept multiple users within the same VR environment thus allowing multiple users to optionally join the same session and review the same case in context of the selected approach and patient anatomy.

In another aspect, the disclosed system may be configured to guide the surgeon during the real operation. This may include, but not be limited to, providing real-time guidance for microsurgical maneuvers. This real-time guidance may be provided via a display device such as a heads up display, navigation station, scope binoculars, and/or exoscope monitor to name a few non-limiting examples. The system may be configured to render the surgical path to the target as determined by the method of the present disclosure. This may be provided in real time so the surgeon can track his or her progress toward the lesion. In another aspect, microscope robotic motion may be used to move the microscope along the surgical path and assist with robotic visualization. In another aspect, the system may be configured to accept user input in the form of voice commands. These commands may be useful, for example, in activating or deactivating different aspects of the superimposed imagery generated by the system and displayed for the care giver. The system may also be configured to accept voice commands activating or deactivating aspects of equipment or tools that may be in use. For example, the system may be configured to activate or deactivate bipolar forceps via voice activation.

In another aspect, the location of the surgical instruments along the path determined by the disclosed method may be identified and the relevant path segments and nearby structures highlighted. The surgical path to the lesion or tumor may be shown to the surgeon by indicating in real time where the surgeon's instruments are relative to the calculated path. The system may also be configured to provide indicators showing deviations from the prescribed path as they occur. In another aspect, hazard zones identified by the disclosed method may be displayed to warn the surgeon if the surgeon is getting too close to them during the procedure.

Specific surgical instruments such as bipolar forceps, hand-held drills, and ultrasonic aspirators, as well as suction devices may be navigated within the 3D space rendered by the disclosed method, and their position in the vicinity of the tumor capsule and segmented critical structures may optionally invoke an alert signal to the surgeon.

In another aspect, the system may be configured to control aspects of the equipment. For example, the system may be configured to adjust the zoom level of a surgical microscope via the pre-configured algorithm based on the real time location of the surgeon along the surgical path.

In another aspect, once the surgical instruments have arrived at the lesion and the surgeon's movements are monitored via real time navigation information, the disclosed system may be configured to provide guidance for the surgeon regarding the most efficient plan to handle/excise the lesion from the Dissection Zone. The Dissection Zone generally refers to the volume of the target lesion or tumor and a 2 cm margin around and just beyond the boundaries of the target in all directions. This dissection zone may be automatically defined by the disclosed system and the segmented structures within this space may be automatically determined based on patient specific imagery as disclosed herein elsewhere.

For example, the disclosed system may provide guidance on the perilesional anatomy (segmented structures around the tumor). In addition, the critical structures that were segmented during previous steps of the disclosed method which are also located within the Dissection Zone may be used to alert the surgeon if intraoperative dissection nears these critical structures that need to be protected.

In another aspect, the system of the present disclosure may be configured to estimate brain shift that may occur during a surgical procedure. For example, the vector of the gravity on the head may be used to offer recommendations in conjunction with path calculations and real-time imagery feedback discussed herein elsewhere. The system may be configured to optionally estimate the amount of brain shift in real time and to further determine areas of the tumor that will be most affected by brain shift.

In another aspect the disclosed system is configured to provide real time information about the location of the tumor in 3D space as the head is immobilized using a clamp assembly or similar device. For example, as the clamp assembly is locked into its final position on the operating room table, real-time feedback about the location of the brain and the tumor or lesion may be obtained. For example, stereotactic data from a navigation device may be obtained along with other data such as pressure sensor data obtained from pressure sensors in the clamp assembly. This data may be aggregated to provide detailed real-time location information about the brain and the tumor. Pressure sensors (e.g. strain gauges) may be incorporated into the clamp assembly and may be configured to provide real time feedback about the risk of slippage of the head relative to the clamp assembly and lack of head fixation relative to contact surfaces (e.g. “pins”) of the clamp. In this context, “contact surfaces” generally refers to predefined regions of the clamp assembly that are physically in contact with the head structure and are arranged and configured to support the head within the clamp assembly. The system may be configured to accept input indicating the pressure exerted by the head on one or more contact surfaces of the clamp assembly. If this pressure starts to change during surgery, and falls outside predetermined threshold values, an alert signal may be generated by the system such as in the form of an activated audible alarm, visible lamp activation, and the like.

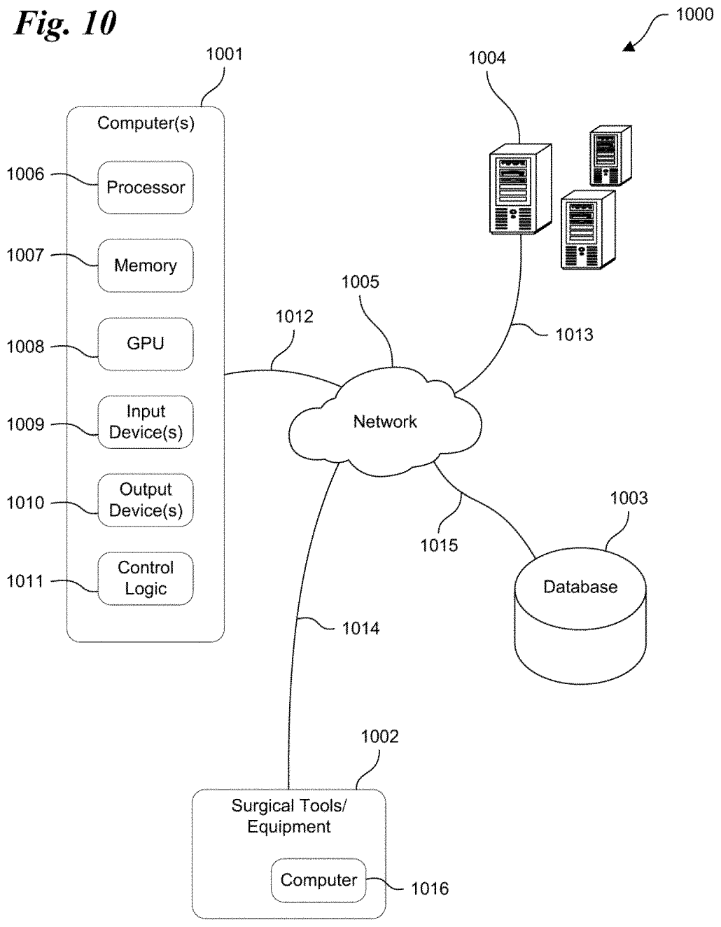

Illustrated at 1000 in FIG. 10 are components that may be useful in the disclosed system. The system may include one or more computers 1001 arranged and configured to perform the disclosed calculations and determinations, to display output, accept input, control other tools, devices, or equipment, and the like, according to the system and method of the present disclosure. A computer 1001 may interact with other components of the system such as via a computer network 1005. Computer 1001 may include a processor 1006 configurable to perform the actions of the disclosed method, or to collaborate with or control other systems or devices to perform these actions. Computer 1001 also optionally includes a memory 1007, a GPU 1008, input devices 1009 (e.g. keyboard, mouse, camera, touchpad, touch screen, stylus, and the like), and output devices 1010 (e.g. monitor, VR glasses, printer, force feedback device, and the like), or any combination thereof. Control logic 1011 may be included and comprise software modules or applications, control circuits, Application Specific Integrated Circuits (ASICs), and the like, or any combination thereof, which may be configured to control the components of computer 1001, or aspects of other disclosed devices, to perform the actions of the disclosed method. Some or all of components 1006-1011, or any combination thereof, may be optionally included in a computer 1001, and others may be included as well.

Other components that may be included in the system of the present disclosure, or may be configured to perform the disclosed methods optionally include surgical tools or equipment 1002 useful for performing surgical procedures, one or more remote computers 1004 for optionally providing additional data processing capabilities, and a database 1003 may be included for optionally providing access to case studies, patient data, information about surgical procedures, and the like. These components may be in communication with one another via network 1005 and communication links 1012-1015. The disclosed components may be physically located in the same enclosure (e.g. part of the same device), in separate enclosures in the same room (e.g. in separate devices), in the same building in different rooms, in different buildings at a location, or physically separated by hundreds or thousands of miles. No particular limitation as to physical location of the components is suggested or imposed by the arrangement shown in FIG. 10.

In another aspect, some of the disclosed components may include other disclosed components such as in the case where one or more pieces of surgical equipment 1002 optionally includes a computer 1016 which may be configured with components like those shown for computers 1001. Other components may be included in the disclosed system and may be useful for enabling the actions the system is configured to take to achieve the outcomes of the disclosed method.

The concepts illustrated and disclosed herein related to a surgical decision support system may be arranged and configured according to any of the following non-limiting numbered examples:

Example 1: A method of providing neurosurgical decision support that includes determining an optimum surgical approach including the best available dissection paths or routes for any target location within the brain.
Example 2: The method of any preceding example including determining the physical attributes of a tumor within a human or animal brain that is at least partially surrounded by the brain.
Example 3: The method of any preceding example including preparing a three-dimensional volumetric model of the tumor according to the physical attributes of the tumor.
Example 4: The method of any preceding example including preparing three-dimensional approach volumes that include volumetric models of regions of the brain occupied by at least a portion of the tumor
Example 5: The method of any preceding example including determining an initial path for each of the approach volumes from an entry point to an end point at the tumor.
Example 6: The method of any preceding example including calculating a ranking for each initial path based on proximity of the initial paths with the approach volumes.
Example 7: The method of any preceding example including determining an optimum surgical path that is based on one or more of the initial paths, wherein the optimum surgical path is selected from among the highest ranked initial paths.
Example 8: The method of any preceding example including subdividing the tumor into discrete sample points in a predetermined grid.
Example 9: The method of any preceding example including obtaining aspects of the approach volumes from a database of predetermined models of regions of a human brain.
Example 10: The method of any preceding example including determining a collection of one or more affected approach volumes occupied by at least a portion of the tumor.
Example 11: The method of any preceding example including determining points on each initial path that are either inside or outside brain tissue.
Example 12: The method of any preceding example including determining the boundaries of regions free of brain tissue that lie between the entry point and the end point.
Example 13: The method of any preceding example including determining at least one entry point with each approach volume, where in each entry point is defined by a three dimensional point in space around the outside of the skull.
Example 14: The method of any preceding example including determining a surgical path for each approach volume that includes the initial craniotomy location and the end point.
Example 15: The method of any preceding example including generating rays originating from each entry point in the direction of the end point, raycasting against a geometric model of the skull, and determining the initial craniotomy location for each approach volume according to the location where each ray of the generated rays intersects the skull.
Example 16: The method of any preceding example including determining one or more exit surfaces defining three dimensional surface meshes that are free of intersections with the approach volumes.
Example 17: The method of any preceding example including determining an exit point along a surgical path where the surgical path leaves the exit surface and enters surrounding approach volumes which may include brain tissue or other portions of the cranium and associated structures within the head and skull base.
Example 18: The method of any preceding example including casting rays from the end point towards the initial craniotomy location, determining a first and second point on the surgical path that are also on the exit surface, assigning first point as the exit point for the surgical path when the first point is closer to the end point than the second point.
Example 19: The method of any preceding example including determining vertices on a pial surface of the brain that are closest to the tumor, and discarding the exit surface when a direct path to the tumor is shorter than a distance between the exit surface and the tumor.
Example 20: The method of any preceding example including generating a curved surgical path for each surgical path.
Example 21: The method of any preceding example including generating a new set of discrete points at uniform intervals along the length of each of the curved surgical paths, and determining when one of the discrete points is inside a portion of the cranium and associated structures such as an approach volume, a hazard zone, or other volumetric data structures.
Example 22: The method of any preceding example including accessing multiple two dimensional cross sectional images of the brain, wherein each two dimensional image has a corresponding z position, and relating x and y values of brain structure represented in the two dimensional images with the corresponding z positions of the two dimensional images to create a volumetric model of cranium and associated structures within the head and skull base, optionally to facilitate the user placement and orientation of anatomical structures in 3D space.
Example 23: The method of any preceding example including determining one or more hazard zones defining three dimensional regions of the brain susceptible to damage by surgical intervention, determining a numerical hazard score for each hazard zone, the hazard score defining a relative danger associated with each hazard zone.
Example 24: The method of any preceding example including determining a hazard score for each initial path by multiplying the hazard score for each hazard zone by a length of the portion of each initial path that is within each hazard zone, and using the hazard score to calculate the ranking for each initial path.
Example 25: The method of any preceding example including determining the length of the initial path, determining a total cumulative distance along the initial path that is within brain tissue, and/or determining the angular alignment of segments of the initial path.
Example 26: The method of any preceding example including accepting input from a user modifying at least one point along the initial paths after the optimum surgical path is determined.
Example 27: The method of any preceding example including accepting input from a user selecting one of the initial paths as the optimum surgical path.
Example 28: The method of any preceding example including displaying the optimum surgical path on a display device, wherein the optimum surgical path is superimposed over an image of the brain.

### Glossary of Definitions and Alternatives

While the invention is illustrated in the drawings and described herein, this disclosure is to be considered as illustrative and not restrictive in character. The present disclosure is exemplary in nature and all changes, equivalents, and modifications that come within the spirit of the invention are included. The detailed description is included herein to discuss aspects of the examples illustrated in the drawings for the purpose of promoting an understanding of the principles of the invention. No limitation of the scope of the invention is thereby intended. Any alterations and further modifications in the described examples, and any further applications of the principles described herein are contemplated as would normally occur to one skilled in the art to which the invention relates. Some examples are disclosed in detail, however some features that may not be relevant may have been left out for the sake of clarity.

Where there are references to publications, patents, and patent applications cited herein, they are understood to be incorporated by reference as if each individual publication, patent, or patent application were specifically and individually indicated to be incorporated by reference and set forth in its entirety herein.

Singular forms “a”, “an”, “the”, and the like include plural referents unless expressly discussed otherwise. As an illustration, references to “a device” or “the device” include one or more of such devices and equivalents thereof.

Directional terms, such as “up”, “down”, “top” “bottom”, “fore”, “aft”, “lateral”, “longitudinal”, “radial”, “circumferential”, etc., are used herein solely for the convenience of the reader in order to aid in the reader's understanding of the illustrated examples. The use of these directional terms does not in any manner limit the described, illustrated, and/or claimed features to a specific direction and/or orientation.

Multiple related items illustrated in the drawings with the same part number which are differentiated by a letter for separate individual instances, may be referred to generally by a distinguishable portion of the full name, and/or by the number alone. For example, if multiple “laterally extending elements” 90A, 90B, 90C, and 90D are illustrated in the drawings, the disclosure may refer to these as “laterally extending elements 90A-90D,” or as “laterally extending elements 90,” or by a distinguishable portion of the full name such as “elements 90”.

Where applicable, actions or steps performed in a method are presumed to be optional and/or executable in any order, not necessarily only in the order described, unless specifically indicated otherwise. Method steps may be presented in a particular order in the accompanying drawings and detailed description, however, this is illustrative rather than restrictive, unless otherwise indicated. Method steps may be performed asynchronously, in parallel, synchronously, or in any suitable combination thereof.

The language used in the disclosure are presumed to have only their plain and ordinary meaning, except as explicitly defined below. The words used in the definitions included herein are to only have their plain and ordinary meaning. Such plain and ordinary meaning is inclusive of all consistent dictionary definitions from the most recently published Webster's and Random House dictionaries. As used herein, the following definitions apply to the following terms or to common variations thereof (e.g., singular/plural forms, past/present tenses, etc.):

“About” with reference to numerical values generally refers to plus or minus 10% of the stated value. For example, if the stated value is 4.375, then use of the term “about 4.375” generally means a range between 3.9375 and 4.8125.

“Activate” generally is synonymous with “providing power to”, or refers to “enabling a specific function” of a circuit or electronic device that already has power.

“And/or” is inclusive here, meaning “and” as well as “or”. For example, “P and/or Q” encompasses, P, Q, and P with Q; and, such “P and/or Q” may include other elements as well.

“Camera” generally refers to a logical, virtual, or physical apparatus or assembly that generates or records images of a viewing area or field-of-view on a medium or in a memory. The images may be still images comprising a single frame or snapshot of the viewing area, or a series of frames recorded over a period of time that may be displayed in sequence to create the appearance of a moving image. Any suitable media may be used to store, reproduce, record, or otherwise maintain the images.

“Communication Link” generally refers to a connection between two or more communicating entities and may or may not include a communications channel between the communicating entities. The communication between the communicating entities may occur by any suitable means. For example, the connection may be implemented as an actual physical link, an electrical link, an electromagnetic link, a logical link, or any other suitable linkage facilitating communication.

In the case of an actual physical link, communication may occur by multiple components in the communication link configured to respond to one another by physical movement of one element in relation to another. In the case of an electrical link, the communication link may be composed of multiple electrical conductors electrically connected to form the communication link.

In the case of an electromagnetic link, the connection may be implemented by sending or receiving electromagnetic energy at any suitable frequency, thus allowing communications to pass as electromagnetic waves. These electromagnetic waves may or may not pass through a physical medium such as an optical fiber, or through free space, or any combination thereof. Electromagnetic waves may be passed at any suitable frequency including any frequency in the electromagnetic spectrum.

A communication link may include any suitable combination of hardware which may include software components as well. Such hardware may include routers, switches, networking endpoints, repeaters, signal strength enters, hubs, and the like.

In the case of a logical link, the communication link may be a conceptual linkage between the sender and recipient such as a transmission station in the receiving station. Logical link may include any combination of physical, electrical, electromagnetic, or other types of communication links.

“Computer” generally refers to any computing device configured to compute a result from any number of input values or variables. A computer may include a processor for performing calculations to process input or output. A computer may include a memory for storing values to be processed by the processor, or for storing the results of previous processing.

A computer may also be configured to accept input and output from a wide array of input and output devices for receiving or sending values. Such devices include other computers, keyboards, mice, visual displays, printers, industrial equipment, and systems or machinery of all types and sizes. For example, a computer can control a network or network interface to perform various network communications upon request. The network interface may be part of the computer or characterized as separate and remote from the computer.

A computer may be a single, physical, computing device such as a desktop computer, a laptop computer, or may be composed of multiple devices of the same type such as a group of servers operating as one device in a networked cluster, or a heterogeneous combination of different computing devices operating as one computer and linked together by a communication network. The communication network connected to the computer may also be connected to a wider network such as the internet. Thus, a computer may include one or more physical processors or other computing devices or circuitry and may also include any suitable type of memory.

A computer may also be a virtual computing platform having an unknown or fluctuating number of physical processors and memories or memory devices. A computer may thus be physically located in one geographical location or physically spread across several widely scattered locations with multiple processors linked together by a communication network to operate as a single computer.

The concept of “computer” and “processor” within a computer or computing device also encompasses any such processor or computing device serving to make calculations or comparisons as part of the disclosed system. Processing operations related to threshold comparisons, rules comparisons, calculations, and the like occurring in a computer may occur, for example, on separate servers, the same server with separate processors, or on a virtual computing environment having an unknown number of physical processors as described above.

A computer may be optionally coupled to one or more visual displays and/or may include an integrated visual display. Likewise, displays may be of the same type, or a heterogeneous combination of different visual devices. A computer may also include one or more operator input devices such as a keyboard, mouse, touch screen, laser or infrared pointing device, or gyroscopic pointing device to name just a few representative examples. Also, besides a display, one or more other output devices may be included such as a printer, plotter, industrial manufacturing machine, 3D printer, and the like. As such, various display, input and output device arrangements are possible.

Multiple computers or computing devices may be configured to communicate with one another or with other devices over wired or wireless communication links to form a network. Network communications may pass through various computers operating as network appliances such as switches, routers, firewalls or other network devices or interfaces before passing over other larger computer networks such as the internet. Communications can also be passed over the network as wireless data transmissions carried over electromagnetic waves through transmission lines or free space. Such communications include using WiFi or other Wireless Local Area Network (WLAN) or a cellular transmitter/receiver to transfer data.

“Contralateral” generally means occurring on, affecting, or acting in conjunction with a part on the opposite side of the body.

“Data” generally refers to one or more values of qualitative or quantitative variables that are usually the result of measurements. Data may be considered “atomic” as being finite individual units of specific information. Data can also be thought of as a value or set of values that includes a frame of reference indicating some meaning associated with the values. For example, the number “2” alone is a symbol that absent some context is meaningless. The number “2” may be considered “data” when it is understood to indicate, for example, the number of items produced in an hour.

Data may be organized and represented in a structured format. Examples include a tabular representation using rows and columns, a tree representation with a set of nodes considered to have a parent-children relationship, or a graph representation as a set of connected nodes to name a few.

The term “data” can refer to unprocessed data or “raw data” such as a collection of numbers, characters, or other symbols representing individual facts or opinions. Data may be collected by sensors in controlled or uncontrolled environments, or generated by observation, recording, or by processing of other data. The word “data” may be used in a plural or singular form. The older plural form “datum” may be used as well.

“Database” also referred to as a “data store”, “data repository”, or “knowledge base” generally refers to an organized collection of data. The data is typically organized to model aspects of the real world in a way that supports processes obtaining information about the world from the data. Access to the data is generally provided by a “Database Management System” (DBMS) consisting of an individual computer software program or organized set of software programs that allow user to interact with one or more databases providing access to data stored in the database (although user access restrictions may be put in place to limit access to some portion of the data).

In another aspect, the DBMS provides various functions that allow entry, storage and retrieval of large quantities of information as well as ways to manage how that information is organized. A database is not generally portable across different DBMSs, but different DBMSs can interoperate by using standardized protocols and languages such as Structured Query Language (SQL), Open Database Connectivity (ODBC), Java Database Connectivity (JDBC), or Extensible Markup Language (XML) to allow a single application to work with more than one DBMS.

In another aspect, a database may implement “smart contracts” which include rules written in computer code that automatically execute specific actions when predetermined conditions have been met and verified. Examples of such actions include, but are not limited to, releasing funds to the appropriate parties, registering a vehicle, sending notifications, issuing a certificate of ownership transfer, and the like. The database may then be updated when the transactions specified in the rules encoded in the smart contract are completely executed. In another aspect, the transaction specified in the rolls may be irreversible and automatically executed without the possibility of manual intervention. In another aspect, only parties specified in the rules of the smart contract who have been granted permission may be notified or allowed to see the results.

Databases and their corresponding database management systems are often classified according to a particular database model they support. Examples include a DBMS that relies on the “relational model” for storing data, usually referred to as Relational Database Management Systems (RDBMS). Such systems commonly use some variation of SQL to perform functions which include querying, formatting, administering, and updating an RDBMS. Other examples of database models include the “object” model, chained model (such as in the case of a “blockchain” database), the “object-relational” model, the “file”, “indexed file” or “flat-file” models, the “hierarchical” model, the “network” model, the “document” model, the “XML” model using some variation of XML, the “entity-attribute-value” model, and others.

Examples of commercially available database management systems include PostgreSQL provided by the PostgreSQL Global Development Group; Microsoft SQL Server provided by the Microsoft Corporation of Redmond, Wash., USA; MySQL and various versions of the Oracle DBMS, often referred to as simply “Oracle” both separately offered by the Oracle Corporation of Redwood City, Calif., USA; the DBMS generally referred to as “SAP” provided by SAP SE of Walldorf, Germany; and the DB2 DBMS provided by the International Business Machines Corporation (IBM) of Armonk, N.Y., USA.

The database and the DBMS software may also be referred to collectively as a “database”. Similarly, the term “database” may also collectively refer to the database, the corresponding DBMS software, and a physical computer or collection of computers. Thus the term “database” may refer to the data, software for managing the data, and/or a physical computer that includes some or all of the data and/or the software for managing the data.

“Display device” generally refers to any device capable of being controlled by an electronic circuit or processor to display information in a visual or tactile. A display device may be configured as an input device taking input from a user or other system (e.g. a touch sensitive computer screen), or as an output device generating visual or tactile information, or the display device may configured to operate as both an input or output device at the same time, or at different times.

The output may be two-dimensional, three-dimensional, and/or mechanical displays and includes, but is not limited to, the following display technologies: Cathode ray tube display (CRT), Light-emitting diode display (LED), Electroluminescent display (ELD), Electronic paper, Electrophoretic Ink (E-ink), Plasma display panel (PDP), Liquid crystal display (LCD), High-Performance Addressing display (HPA), Thin-film transistor display (TFT), Organic light-emitting diode display (OLED), Surface-conduction electron-emitter display (SED), Laser TV, Carbon nanotubes, Quantum dot display, Interferometric modulator display (IMOD), Swept-volume display, Varifocal mirror display, Emissive volume display, Laser display, Holographic display, Light field displays, volumetric display, Ticker tape, Split-flap display, Flip-disc display (or flip-dot display), Rollsign, mechanical gauges with moving needles and accompanying indicia, Tactile electronic displays (aka refreshable Braille display), Optacon displays, or any devices that either alone or in combination are configured to provide visual feedback on the status of a system, such as the “check engine” light, a “low altitude” warning light, an array of red, yellow, and green indicators configured to indicate a temperature range.

“Graphics Processing Unit” or “GPU” generally refers to a specialized electronic circuit designed to manipulate and alter memory to accelerate the creation of images in a frame buffer intended for output to a display device. GPUs are optimized to manipulate computer graphics and perform image processing. Many GPU's have an internal parallel structure that makes them more efficient than general-purpose Central Processing Units (CPUs) for algorithms that process large blocks of data in parallel. This is especially true for data mapped in three-dimensional, or two-dimensional space, and/or data presented in single or multi-dimensional vector or matrix formats.

In some instances, a GPU may be useful for making calculations that do not result in a graphical output on a display device. For example, many deep learning, neural network, or other artificial intelligence algorithms perform calculations on data that is presented using multidimensional matrices. GPU's are generally optimized for these types of calculation and thus may be useful for performing calculations relevant to machine implemented automatic decision-making algorithms with or without any resulting graphical output.

A GPU can be present on a separate video card, embedded on a motherboard, or embedded on a CPU die. Thus GPUs are useful in many different computing devices such as in embedded systems circuits, mobile phones, personal computers, workstations, and game consoles.

“Input Device” generally refers to any device coupled to a computer that is configured to receive input and deliver the input to a processor, memory, or other part of the computer. Such input devices can include keyboards, mice, trackballs, touch sensitive pointing devices such as touchpads, or touchscreens. Input devices also include any sensor or sensor array for detecting environmental conditions such as temperature, light, noise, vibration, humidity, and the like.

“Interhemispheric” generally means extending between hemispheres, such as between the two hemispheres of the brain.

“Ipsilateral” generally means belonging to or occurring on the same side of the body.

“Lesion” generally refers to a region in an organ or tissue which has suffered damage through injury or disease, such as a wound, ulcer, abscess, or tumor.

“Memory” generally refers to any storage system or device configured to retain data or information. Each memory may include one or more types of solid-state electronic memory, magnetic memory, or optical memory, just to name a few. Memory may use any suitable storage technology, or combination of storage technologies, and may be volatile, nonvolatile, or a hybrid combination of volatile and nonvolatile varieties. By way of non-limiting example, each memory may include solid-state electronic Random Access Memory (RAM), Sequentially Accessible Memory (SAM) (such as the First-In, First-Out (FIFO) variety or the Last-In-First-Out (LIFO) variety), Programmable Read Only Memory (PROM), Electronically Programmable Read Only Memory (EPROM), or Electrically Erasable Programmable Read Only Memory (EEPROM).

Memory can refer to Dynamic Random Access Memory (DRAM) or any variants, including static random access memory (SRAM), Burst SRAM or Synch Burst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (REDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM).

Memory can also refer to non-volatile storage technologies such as non-volatile read access memory (NVRAM), flash memory, non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Domain Wall Memory (DWM) or “Racetrack” memory, Nano-RAM (NRAM), or Millipede memory. Other non-volatile types of memory include optical disc memory (such as a DVD or CD ROM), a magnetically encoded hard disc or hard disc platter, floppy disc, tape, or cartridge media. The concept of a “memory” includes the use of any suitable storage technology or any combination of storage technologies.

“Module” or “Engine” generally refers to a collection of computational or logic circuits implemented in hardware, or to a series of logic or computational instructions expressed in executable, object, or source code, or any combination thereof, configured to perform tasks or implement processes. A module may be implemented in software maintained in volatile memory in a computer and executed by a processor or other circuit. A module may be implemented as software stored in an erasable/programmable nonvolatile memory and executed by a processor or processors. A module may be implanted as software coded into an Application Specific Information Integrated Circuit (ASIC). A module may be a collection of digital or analog circuits configured to control a machine to generate a desired outcome.

Modules may be executed on a single computer with one or more processors, or by multiple computers with multiple processors coupled together by a network. Separate aspects, computations, or functionality performed by a module may be executed by separate processors on separate computers, by the same processor on the same computer, or by different computers at different times.

“Multiple” as used herein is synonymous with the term “plurality” and refers to more than one, or by extension, two or more.

“Network” or “Computer Network” generally refers to a telecommunications network that allows computers to exchange data. Computers can pass data to each other along data connections by transforming data into a collection of datagrams or packets. The connections between computers and the network may be established using either cables, optical fibers, or via electromagnetic transmissions such as for wireless network devices.

Computers coupled to a network may be referred to as “nodes” or as “hosts” and may originate, broadcast, route, or accept data from the network. Nodes can include any computing device such as personal computers, phones, servers as well as specialized computers that operate to maintain the flow of data across the network, referred to as “network devices”. Two nodes can be considered “networked together” when one device is able to exchange information with another device, whether or not they have a direct connection to each other.

Examples of wired network connections may include Digital Subscriber Lines (DSL), coaxial cable lines, or optical fiber lines. The wireless connections may include BLUETOOTH, Worldwide Interoperability for Microwave Access (WiMAX), infrared channel or satellite band, or any wireless local area network (Wi-Fi) such as those implemented using the Institute of Electrical and Electronics Engineers' (IEEE) 802.11 standards (e.g. 802.11(a), 802.11(b), 802.11(g), or 802.11(n) to name a few). Wireless links may also include or use any cellular network standards used to communicate among mobile devices including 1G, 2G, 3G, or 4G. The network standards may qualify as 1G, 2G, etc. by fulfilling a specification or standards such as the specifications maintained by International Telecommunication Union (ITU). For example, a network may be referred to as a “3G network” if it meets the criteria in the International Mobile Telecommunications-2000 (IMT-2000) specification regardless of what it may otherwise be referred to. A network may be referred to as a “4G network” if it meets the requirements of the International Mobile Telecommunications Advanced (IMTAdvanced) specification. Examples of cellular network or other wireless standards include AMPS, GSM, GPRS, UMTS, LTE, LTE Advanced, Mobile WiMAX, and WiMAX-Advanced.

Cellular network standards may use various channel access methods such as FDMA, TDMA, CDMA, or SDMA. Different types of data may be transmitted via different links and standards, or the same types of data may be transmitted via different links and standards.

The geographical scope of the network may vary widely. Examples include a body area network (BAN), a personal area network (PAN), a low power wireless Personal Area Network using IPv6 (6LoWPAN), a local-area network (LAN), a metropolitan area network (MAN), a wide area network (WAN), or the Internet.

A network may have any suitable network topology defining the number and use of the network connections. The network topology may be of any suitable form and may include point-to-point, bus, star, ring, mesh, or tree. A network may be an overlay network which is virtual and is configured as one or more layers that use or “lay on top of” other networks.

A network may utilize different communication protocols or messaging techniques including layers or stacks of protocols. Examples include the Ethernet protocol, the internet protocol suite (TCP/IP), the ATM (Asynchronous Transfer Mode) technique, the SONET (Synchronous Optical Networking) protocol, or the SDE1 (Synchronous Digital Elierarchy) protocol. The TCP/IP internet protocol suite may include application layer, transport layer, internet layer (including, e.g., IPv6), or the link layer.

“Octree” generally refers to a tree data structure in which each internal node has exactly eight children. For example, an octrees may be used to partition a three-dimensional space by recursively subdividing it into eight octants. Octrees are often used in 3D graphics and 3D game engines where volumetric modeling is used to create a virtual representation of a real world environment.

“Optionally” as used herein means discretionary; not required; possible, but not compulsory; left to personal choice.

“Output Device” generally refers to any device or collection of devices that is controlled by computer to produce an output. This includes any system, apparatus, or equipment receiving signals from a computer to control the device to generate or create some type of output. Examples of output devices include, but are not limited to, screens or monitors displaying graphical output, any projector a projecting device projecting a two-dimensional or three-dimensional image, any kind of printer, plotter, or similar device producing either two-dimensional or three-dimensional representations of the output fixed in any tangible medium (e.g. a laser printer printing on paper, a lathe controlled to machine a piece of metal, or a three-dimensional printer producing an object). An output device may also produce intangible output such as, for example, data stored in a database, or electromagnetic energy transmitted through a medium or through free space such as audio produced by a speaker controlled by the computer, radio signals transmitted through free space, or pulses of light passing through a fiber-optic cable.

“Personal computing device” generally refers to a computing device configured for use by individual people. Examples include mobile devices such as Personal Digital Assistants (PDAs), tablet computers, wearable computers installed in items worn on the human body such as in eyeglasses, watches, laptop computers, portable music/video players, computers in automobiles, or cellular telephones such as smart phones. Personal computing devices can be devices that are typically not mobile such as desk top computers, game consoles, or server computers. Personal computing devices may include any suitable input/output devices and may be configured to access a network such as through a wireless or wired connection, and/or via other network hardware.

“Pia” generally refers to the delicate innermost membrane enveloping the brain and spinal cord.

“Pial Surface” generally refers to a surface representing the boundary between grey matter and cerebrospinal fluid, typically as reconstructed from neuroimaging data. The pial surface roughly corresponds to the position of the pia mater.

“Portion” means a part of a whole, either separated from or integrated with it.

“Predominately” as used herein is synonymous with greater than 50%.

“Processor” generally refers to one or more electronic components configured to operate as a single unit configured or programmed to process input to generate an output. Alternatively, when of a multi-component form, a processor may have one or more components located remotely relative to the others. One or more components of each processor may be of the electronic variety defining digital circuitry, analog circuitry, or both. In one example, each processor is of a conventional, integrated circuit microprocessor arrangement, such as one or more PENTIUM, i3, i5 or i7 processors supplied by INTEL Corporation of Santa Clara, Calif., USA. Other examples of commercially available processors include but are not limited to the X8 and Freescale Coldfire processors made by Motorola Corporation of Schaumburg, Ill., USA; the ARM processor and TEGRA System on a Chip (SoC) processors manufactured by Nvidia of Santa Clara, Calif., USA; the POWER7 processor manufactured by International Business Machines of White Plains, N.Y., USA; any of the FX, Phenom, Athlon, Sempron, or Opteron processors manufactured by Advanced Micro Devices of Sunnyvale, Calif., USA; or the Snapdragon SoC processors manufactured by Qalcomm of San Diego, Calif., USA.

A processor also includes Application-Specific Integrated Circuit (ASIC). An ASIC is an Integrated Circuit (IC) customized to perform a specific series of logical operations is controlling a computer to perform specific tasks or functions. An ASIC is an example of a processor for a special purpose computer, rather than a processor configured for general-purpose use. An application-specific integrated circuit generally is not reprogrammable to perform other functions and may be programmed once when it is manufactured.

In another example, a processor may be of the “field programmable” type. Such processors may be programmed multiple times “in the field” to perform various specialized or general functions after they are manufactured. A field-programmable processor may include a Field-Programmable Gate Array (FPGA) in an integrated circuit in the processor. FPGA may be programmed to perform a specific series of instructions which may be retained in nonvolatile memory cells in the FPGA. The FPGA may be configured by a customer or a designer using a hardware description language (HDL). In FPGA may be reprogrammed using another computer to reconfigure the FPGA to implement a new set of commands or operating instructions. Such an operation may be executed in any suitable means such as by a firmware upgrade to the processor circuitry.

Just as the concept of a computer is not limited to a single physical device in a single location, so also the concept of a “processor” is not limited to a single physical logic circuit or package of circuits but includes one or more such circuits or circuit packages possibly contained within or across multiple computers in numerous physical locations. In a virtual computing environment, an unknown number of physical processors may be actively processing data, the unknown number may automatically change over time as well.

The concept of a “processor” includes a device configured or programmed to make threshold comparisons, rules comparisons, calculations, or perform logical operations applying a rule to data yielding a logical result (e.g. “true” or “false”). Processing activities may occur in multiple single processors on separate servers, on multiple processors in a single server with separate processors, or on multiple processors physically remote from one another in separate computing devices.

“Retain” generally refers to the act of keeping possession or use of something; the act of remembering by keeping in mind or memory, such as in the context of storing in a computer memory whether in volatile, nonvolatile, or other memory; or to hold one object secure or intact relative to another such as in the physical sense via a fastening member or material.

“Raycasting” generally refers to a process for programmatically determining what lines or surfaces can be intersected (i.e. are “visible”) from a given viewpoint with a particular field of view. It involves determining a ray defined by a starting point P, and a vector V, and then traveling from P in either 3D or 2D space in the direction of vector V until a surface (in 3D space) or an intersecting line (in 2D space) is encountered. This operation may be performed for multiple vectors V for a given view point P thus making it possible to determine the surfaces or lines present for a given field of view with a high degree of precision which depends on the number of ray calculations performed.

Raycasting involves determining the first object surface or line the ray hits, if any, and optionally calculating a vector normal to the surface encountered at the point of intersection. Ray casting generally differs from raytracing in that ray casting generally does not involve recursively determining secondary rays resulting from these initial collision calculations.

“Rule” generally refers to a conditional statement with at least two outcomes. A rule may be compared to available data which can yield a positive result (all aspects of the conditional statement of the rule are satisfied by the data), or a negative result (at least one aspect of the conditional statement of the rule is not satisfied by the data). One example of a rule is shown below as pseudo code of an “if/then/else” statement that may be coded in a programming language and executed by a processor in a computer:

```
if(clouds.areGrey() and
(clouds.numberOfClouds > 100)) then {
  prepare for rain;
} else {
  Prepare for sunshine;
}
```

“Short Message Service (SMS)” generally refers to a text messaging service component of phone, Web, or mobile communication systems. It uses standardized communications protocols to allow fixed line or mobile phone devices to exchange short text messages. Transmission of short messages between a Short Message Service Center (SMSC) and personal computing device is done whenever using the Mobile Application Part (MAP) of the SS7 protocol. Messages payloads may be limited by the constraints of the signaling protocol to precisely 140 octets (140 octets*8 bits/octet=1120 bits). Short messages can be encoded using a variety of alphabets: the default GSM 7-bit alphabet, the 8-bit data alphabet, and the 16-bit UCS-2 alphabet. Depending on which alphabet the subscriber has configured in the handset, this leads to the maximum individual short message sizes of 160 7-bit characters, 140 8-bit characters, or 70 16-bit characters.

“Suboccipital” generally means below the occipital bone.

“Supracerebellar” generally means located on or above the upper surface of the cerebellum.

“Viewing Area”, “Field of View”, or “Field of Vision” is the extent of the observable world that is seen at any given moment. In case of optical instruments, cameras, or sensors, it is a solid angle through which a detector is sensitive to electromagnetic radiation that includes light visible to the human eye, and any other form of electromagnetic radiation that may be invisible to humans.

“Voxel” generally refers to a unit of graphic information that defines a point in three-dimensional space. It is analogous to a pixel (picture element) which defines a point in two dimensional space with its X and Y coordinates. For a voxel, a third Z coordinate is typically used. As with pixels, voxels generally do not have their position (i.e. coordinates) explicitly encoded with their values. Instead, rendering systems infer the position of a voxel based upon its position relative to other voxels (i.e., its position in the data structure that makes up a single volumetric image).

In another aspect, a voxel refers to an image of a three-dimensional space region limited by given sizes, which has its own nodal point coordinates in an accepted coordinate system, its own form, its own state parameter that indicates its belonging to some modeled object, and has properties of a modeled region.

What is claimed is:

1. A method, comprising:
determining physical attributes of a tumor within a human or animal using one or more processors of one or more computers, wherein the tumor is at least partially surrounded by the brain or other organs;
preparing a three-dimensional volumetric model of the tumor according to the physical attributes of the tumor using the one or more processors;
preparing three-dimensional approach volumes using the one or more processors, wherein the approach volumes include volumetric models of regions of the brain occupied by at least a portion of the tumor;
determining an initial path for each of the approach volumes from an entry point to an end point at the tumor using the one or more processors;
calculating a ranking for each initial path based on proximity of the initial paths with the approach volumes using the one or more processors; and
determining an optimum surgical path that is based on one or more of the initial paths using the one or more processors, wherein the optimum surgical path is selected from among highest ranked initial paths.

2. The method of claim 1, comprising:
subdividing the tumor into discrete sample points in a predetermined grid using the one or more processors.

3. The method of claim 1, comprising:
obtaining aspects of the approach volumes from a database of predetermined models of regions of a human brain.

4. The method of claim 1, comprising:
determining a collection of one or more affected approach volumes occupied by at least a portion of the tumor using the one or more processors.

5. The method of claim 1, comprising:
using the one or more processors to determine points on each initial path that are either inside or outside brain tissue.

6. The method of claim 1, comprising:
using the one or more processors to determine boundaries of regions free of brain tissue that lie between the entry point and the end point.

7. The method of claim 1, comprising:
determining at least one entry point with each approach volume, where in each entry point is defined by a three dimensional point in space outside of skull structures that contain the brain.

8. The method of claim 7, comprising:
determining a surgical path for each approach volume that includes an initial craniotomy location and the end point, using the one or more processors.

9. The method of claim 8, comprising:
generating rays originating from each entry point in a direction toward the end point using the one or more processors;
raycasting against a geometric model of the skull structures using the one or more processors; and
determining the initial craniotomy location for each approach volume according to a location where each ray of the generated rays intersects the skull structures.

10. The method of claim 1, comprising:
determining one or more exit surfaces defining three dimensional surface meshes that are free of intersections with brain tissue using the one or more processors.

11. The method of claim 10, comprising:
determining an exit point along a surgical path where the surgical path leaves a corresponding exit surface and enters surrounding brain tissue using the one or more processors.

12. The method of claim 11, wherein determining the exit point comprises:
casting rays from the end point towards the initial craniotomy location using the one or more processors;
determining a first and second point on the surgical path that are also on the exit surface; and
assigning first point as the exit point for the surgical path when the first point is closer to the end point than the second point.

13. The method of claim 10, wherein determining the exit point comprises:
using the one or more processors to determine vertices on a pial surface of the brain that are closest to the tumor; and
discarding the exit surface when a direct path to the tumor is shorter than a distance between the exit surface and the tumor.

14. The method of claim 9, comprising:
using the one or more processors to generate a curved surgical path for each surgical path.

15. The method of claim 14, comprising:
generating a new set of discrete points at uniform intervals along a length of each of the curved surgical paths using the one or more processors; and
using the one or more processors to determine when one of the discrete points is inside a hazard zone.

16. The method of claim 1, comprising:
accessing multiple two dimensional cross sectional images of the brain, wherein each two dimensional image has a corresponding z position using the one or more processors; and
relating x and y values of brain structure represented in the two dimensional images with the corresponding z positions of the two dimensional images to create a volumetric model of a cranium and associated structures within a head and skull base using the one or more processors.

17. The method of claim 1, comprising:
determining one or more hazard zones defining three dimensional regions of the brain susceptible to damage by surgical intervention using the one or more processors; and
determining a numerical hazard score for each hazard zone, the numerical hazard score defining a relative danger associated with each hazard zone using the one or more processors.

18. The method of claim 17, comprising:
determining a hazard score for each initial path by multiplying the hazard score for each hazard zone by a length of the portion of each initial path that is within each hazard zone using the one or more processors; and
using the hazard score to calculate the ranking for each initial path.

19. The method of claim 1, wherein calculating the ranking for each initial path comprises:
determining a length of the initial path;
determining a total cumulative distance along the initial path that is within brain tissue; and/or
determining an angular alignment of the approach path relative to the longitudinal axis of the tumor.

20. The method of claim 1, comprising:
accepting input from a user modifying at least one point along the initial paths after the optimum surgical path is determined.

21. The method of claim 1, comprising:
accepting input from a user selecting one of the initial paths as the optimum surgical path.

22. The method of claim 1, comprising:
displaying the optimum surgical path on a display device, wherein the optimum surgical path is superimposed over an image of the brain.
